(19) United States
(12) Patent Application Publication
(10) Pub. No.: US 2022/0047702 A1
(43) Pub. Date: Feb. 17, 2022

(54) TARGETED MAGNETIC VEHICLES AND METHOD OF USING THE SAME

(71) Applicants: HADASIT MEDICAL RESEARCH SERVICES AND DEVELOPMENT LTD., Jerusalem (IL); BAR-ILAN UNIVERSITY, Ramat Gan (IL)

(72) Inventors: Yoav MINTZ, Jerusalem (IL); Nikolai KUNICHER, Pardesia (IL); Koby GOREN, Mevaseret Zion (IL); Shlomo MARGEL, Rehovot (IL)

(21) Appl. No.: 17/460,235

(22) Filed: Aug. 29, 2021

Related U.S. Application Data
(63) Continuation of application No. PCT/IL2020/050225, filed on Feb. 27, 2020.
(60) Provisional application No. 62/811,657, filed on Feb. 28, 2019.

(51) Int. Cl. A61K 39/395; A61K 41/00; A61K 31/513; A61K 9/51; A61P 35/00

(52) U.S. Cl. CPC A61K 39/3955; A61K 41/00; A61P 35/00; A61K 9/5169; A61K 9/5115; A61K 31/513

(57) ABSTRACT

The present invention is directed to a nanoparticle including: a core of a metal chelating polymer, wherein the core is coated with at least two layers of magnetic metal oxide, which are further coated with a protein layer, and at least one active agent bound within or to the nanoparticle, methods for directing or targeting the nanoparticle via a magnetic field to a target tissue, and a computer program for efficiently generating the proper magnetic field for driving the nanoparticle to or into a given target tissue.

### CROSS REFERENCE TO RELATED APPLICATIONS

This application is a Bypass Continuation of PCT Patent Application No. PCT/IL2020/050225 having International filing date of Feb. 27, 2020 which claims the benefit of priority under 35 U.S.C. § 119(e) of U.S. Provisional Patent Application No. 62/811,657, titled “TARGET MAGNETIC VEHICLES AND METHOD OF USING THE SAME”, filed on Feb. 28, 2019. The contents of the above applications are all incorporated herein by reference as is fully set forth herein in their entirety.

### FIELD OF INVENTION

The present invention is in the field of targeting pharmaceutical vehicles by a magnetic field.

### BACKGROUND OF THE INVENTION

The treatment of cancer is the subject of many research projects. Current cancer treatments are associated with side effects such as: nausea, vomiting, hair loss, liver toxicity etc. These side effects are the result of the inability to treat the tumor locally, instead toxic treatment is exposed to many tissue including healthy tissues and therefore the entire body may be affected.

Non resectable tumors such as rectal cancer and pancreatic cancer have no effective treatment, thereby leading to high mortality rate. Patient afflicted with such cancers may benefit from targeted or local administration of anti-tumor drugs which can effectively decrease the tumor volume, increase the survival rate and may be even serve as neoadjuvant therapy leading these tumors to be resectable.

Nanoparticles are spherical particles in sizes ranging from a few nanometers up to 0.1 μm. Polymeric nano-scaled particles of narrow size distribution are commonly formed by controlled precipitation methods or heterogeneous polymerization techniques, e.g., by optimal emulsion or inverse emulsion polymerization methods. Properties of solid materials undergo drastic changes when their dimensions are reduced to the nanometer size regime. It is important to keep in mind that the smaller the particles are, the larger portion of their constituent atoms is located at the surface. Nanoparticles, particularly in sizes below ca. 20 nm, predominantly exhibit surface and interface phenomena that are not observed in bulk materials, e.g., lower melting and boiling points, lower sintering temperature and reduced flow resistance.

In view of their spherical shape and high surface area, nano-scaled particles may provide neat solutions to a variety of problems in materials science, such as composite materials, catalysis, three dimensional structures and photonic uses, and can further be used in biomedical applications such as specific cell labeling and separation, cell growth, affinity chromatography, specific blood purification by hemoper fusion, drug delivery and controlled release (Bockstaller et al., 2003; Hergt et al., 2004; Margel et al., 1999). Each application requires polymeric nanoparticles of different optimal physical and chemical properties. The synthesis and use of numerous types of nano-scaled particles of different surface chemistry, e.g., variety of surface functional groups such as hydroxyl, carboxyl, pyridine, amide, aldehyde and phenyl chloromethyl, have already been described (Margel et al, 1999). Such nanoparticles have been designed for various industrial and medical applications, e.g., enzyme immobilization, oligonucleotide and peptide synthesis, drug delivery, specific cell labeling and separation, medical imaging, biological glues and flame retardant polymers (Bunker et al, 1994; Szymonifka and Chapman, 1995; Margel et al, 1999; WO 2004/045494; Galperin et al, 2007).

Of particular interest are particles with magnetic properties, which are usually used for separation of the particles and/or their conjugates from undesired compounds via a magnetic field. Due to their magnetic properties, these particles have several additional significant applications such as magnetic recording, magnetic sealing, electromagnetic shielding and biomedical applications. Magnetic iron oxide, i.e., magnetite and maghemite, nanoparticles are the main particles that have been investigated for biomedical applications, e.g., magnetic hyperthermia, magnetic drug targeting, magnetic cell separation and as MRI contrast agents (Lacoste et al, 1993; Green-Sadan et al, 2005; Leemputten and Horisberger, 1974; Hergt et al, 2004). Magnetic iron oxides nanoparticles are non-toxic and biodegradable and have already been approved for clinical use as MRI contrast agents. These nanoparticles are usually prepared by adding to an aqueous solution containing stoichiometric concentrations of ferrous and ferric ions, and a polymeric stabilizer such as dextran, wherein a base, e.g., NaOH or ammonia, is added until basic pH (usually above 8.0) is reached. The obtained coated magnetic iron oxide nanoparticles are than washed by different ways, e.g., by magnetic columns or dialysis. Extensive efforts to synthesize efficient iron oxide magnetic nanoparticles have been carried out in the last several years; however, most of these nanoparticles suffer from major disadvantages such as broad size distribution that is considered to be toxic for in vivo medical applications, iron ions leaching and instability towards agglutination processes.

WO 99/062079 and corresponding EP 1088315B1 of the same Applicant, herewith incorporated by reference in their entirety as if fully disclosed herein, disclose new uniform magnetic gelatin/iron oxide composite nanoparticles, formed by controlled nucleation of iron oxide onto an iron ion chelating polymer, e.g., gelatin, dissolved in an aqueous solution, followed by stepwise growth of thin layers of iron oxide films onto the gelatin/iron oxide nuclei.

### SUMMARY OF THE INVENTION

In some embodiments, the present invention provides a nanoparticle comprising: a core comprising a metal chelating polymer, the metal chelating polymer is coated with at least two layers of magnetic metal oxide, at least two layers of magnetic metal oxide are further coated with a protein layer, and at least one active agent bound within the nanoparticle.

The polymer may include functional groups capable of binding metal ions such as: amino, hydroxyl, carboxylate, —SH, ether, imine, phosphate or sulfide groups. The polymer may be selected from: gelatin, polymethylenimine, chitosan or polylysine.

In a further embodiment, the at least one active agent is selected from: a chemotherapeutic agent, an immunotherapeutic agent, a fluorescent dye, a contrast agent, a peptide, a peptidomimetic, a polypeptide a small molecule, or any combination thereof.

In a further embodiment, the nanoparticle further comprises at least one compound: (a) physically or covalently bound to the outer surface of the magnetic metal oxide, (b) physically or covalently bound to the outer surface of the protein layer, (c) physically or covalently bound to the metal chelating polymer, or a combination thereof.

In a further embodiment, provided herein is a pharmaceutical composition comprising a nanoparticle as described herein.

In a further embodiment, provided herein is a method for contacting superparamagnetic nanoparticle or the nanoparticle as described herein with, delivering superparamagnetic nanoparticle or the nanoparticle as described herein to, or targeting superparamagnetic nanoparticle or the nanoparticle as described herein, a target tissue in a subject, comprising administering a superparamagnetic nanoparticle or the nanoparticle as described herein, to the subject and applying a magnetic field, to induce the superparamagnetic nanoparticle or the nanoparticle as described herein to contact with or penetrate into the target tissue, thereby contacting, delivering or targeting the nanoparticle.

In a further embodiment, provided herein is a kit or a system comprising a pharmaceutical composition comprising a superparamagnetic nanoparticle or a nanoparticle as described herein and a magnetic field generating magnet.

In a further embodiment, provided herein is a computer program comprising the step of: generating planning data for contacting a target tissue with a a superparamagnetic nanoparticle or a nanoparticle as described herein; wherein said planning data comprises planning or setting: A. the amount nanoparticles comprising a metal chelating polymer, said metal chelating polymer is coated with at least two layers of magnetic metal oxide, said at least two layers of magnetic metal oxide are further coated with a protein layer, and at least one active agent bound within said nanoparticle; B. the average number of said layers of magnetic metal oxide; C. the dose of said at least one active agent; D. the location magnetic field generator; E. the magnetic field strength; F. the magnetic field gradient; or any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. is a bar graph showing cell viability test (XTT) on Gly-PEG-IO, AV-PEG-IO, AV/IO, IO nanoparticles (NPs) and free Avastin on HUVEC cells after 48 h treatment. [Legend: Gly-IO/HSA (human serum albumin), AV-IO/HSA, AV-physically conjugated IO/HSA, IO NPs].

FIGS. 2A and 2B. are bar graphs showing cell viability test (XTT) of the 5FU-physically conjugated IO/HSA NPs and control IO/HSA NPs on SW620 (human colon adenocarcinoma) and A172 (human neuroblastoma) cell lines.

FIGS. 3A and 3B. are graphs showing intracellular fluorescence of SW620 (human colon adenocarcinoma) and A172 (human neuroblastoma) cells treated with 4×, 6× or 8× IONPs. The figures comparing positive cell uptake as a function of x, which represents the number of coatings on IONPs.

FIGS. 4A and 4B. are graphs showing the hysteresis behavior of 6× IO NP, IO/HSA and IO/HSA-SFU.

FIG. 5. is a graph showing the EDAX of the 5FU-physically conjugated IO/HSA, indicating 20.6 w % F.

FIG. 6. histochemistry micrographs showing iron staining (blue) of tumors treated systemically with 4×IONP-HSA and exposed to a magnetic field for (A-D) 4 hours and (E) 24 hours. (B, D) ×20 magnification of A and C respectively.

FIG. 7. depicts the in-vivo experimental design—A) N35 neodymium magnets were embedded inside a plastic container containing four ears used for suturing to mice skin. B) The 5FU-IONP or IONP were injected underneath the tumors at days 0 and 5 followed by C) application of the magnet embedded in its plastic container or the plastic container with no magnet. The mice were euthanized at day 7, and the tumors, liver, and spleen were extracted for histological analysis.

FIG. 8. are graphs showing the antitumor activity of 5FU-IONP -A, B) 5FU-IONP (experimental) or IONP (control) was injected underneath the tumor at days 0 and 5. The tumors were exposed to a magnetic field for 4 hours following each injection. A) The average tumor volume of the experimental group was significantly lower compared to the control group treated with IONP starting from day 4 and compared to the control group treated with 5FU-IONP without exposure to the magnetic field at days 6 and 7 (n=5 per group). B) The growth rate of the tumors presented as the ratio between the tumor volume at each day and at day 0. The experimental group showed a significantly lower growth rate of the tumors compared to both control groups (n=5 per group). C, D) 5FU were injected IP in three consecutive days starting at day 3 with no significant difference in C) the tumor volume and D) tumor growth rate as opposed to the control group injected with normal saline (placebo, n=10 per group). Black arrow marks the day of injection, *P<0.05, **P<0.01, ***P<0.001 (RM two way ANOVA with a confidence interval of 95% using post hoc A, B) Dunnet's analysis or C, D) Sidak's analysis for multiple comparisons.

FIG. 9. histochemistry micrographs showing Iron staining (blue) of tumors treated locally with 4×IONP-HSA and (A-D) exposed to a magnetic field for 2 hours. Blue staining was evident (A, B) inside the tumor (B×20 Magnification of A) and (C, D) the area surrounding the tumor was highly stained. (E, F) No staining was found inside the tumors were not exposed to the magnetic field. Blue staining was evident only underneath the tumor in the area of injection.

FIG. 10. histochemistry micrographs showing exposure of 6×IONP-5FU to a magnetic field directed the nanoparticle inside the tumor—6×IONP-5FU were injected underneath the tumor (A) with or (B) without magnetic field application for 4 hours. Prussian blue staining showed that the application of a magnetic field reduced the clearance of the nanoparticles. More staining is clearly evident inside the tumors which exposed to the magnetic field.

FIG. 11. histochemistry micrographs showing iron staining of the liver excised from mice treated with (A) systemically administered 4×IONP-HSA and exposed to a magnetic field for 24 hours; (B-E) locally administered 6×IONP-5FU exposed to a magnetic field for (B) 4 hours, or (C) 6 hours, and (D, E) without magnetic field exposure. Local administration of 6×IONP-5FU yielded weaker iron staining in the liver compared to systemic administration of 4×IONP-HSA.

FIG. 12. histochemistry micrographs showing iron staining coverage area in the spleen of the mice that treated with 6×IONP-5FU locally underneath the tumor and exposed to a magnetic field for 4 hours or 6 hours. (A, C) The plastic chassis without a magnet (control) or (B, D) with a magnet was sutured on top of the tumors. (E) Quantification of the blue staining showed significantly lower iron staining coverage area in the spleen of mice exposed to an external magnetic field. * P<0.05, **P<0.01.

FIG. 13 are graphs showing tumor volume as affected by targeting Bev. (A) systemic or local administration of 10 mg/kg Bev without local tumoral magnetic field had no effect on tumor growth, while (B) administration of 3 mg/kg Bev with local tumoral magnetic field inhibited tumor growth. Only tumors treated with SPION-BEV+magnet had statistically stopped growing.

FIG. 14 is a bar graph of tumor sections areas stained for CD31-vascular coverage area of tumor. (right) % of CD31 tumor area treated with SPION-Bev under magnetic field and (left) % of CD31 tumor area treated with SPION-Bev without magnetic field.

### DETAILED DESCRIPTION OF THE INVENTION

In some embodiments, the present invention provides a particle or a nanoparticle comprising: a core comprising a metal chelating polymer, wherein the metal chelating polymer is coated with at least two layers of magnetic metal oxide, wherein at least two layers of magnetic metal oxide are further coated with a protein layer or a shell, and at least one active agent is physically attached or bound or entrapped within the nanoparticle or to the shell. In some embodiments, the present invention provides a nanoparticle comprising: a core comprising a metal chelating polymer, said metal chelating polymer is coated with at least two layers of magnetic metal oxide, wherein the at least two layers of magnetic metal oxide are further coated with a protein layer, the nanoparticle comprises bevacizumab (Bev), Fluorouracil (5-FU), or a combination thereof. In one embodiment, bevacizumab (Bev), Fluorouracil (5-FU), or a combination thereof is physically attached or bound or entrapped within the nanoparticle or to the shell.

In one embodiment, “particle” comprises: a microparticle, a nanoparticle or both. In some embodiments, “entrapped” comprises contained in-between layer of the nanoparticle. In some embodiments, bound is bound to the: metal chelating polymer, metal oxide layer, protein layer/shell or any combination thereof. In some embodiments, bound is physical binding. In some embodiments, bound comprises a covalent bond. In some embodiments, bound comprises chemical attraction. In one embodiment, a nanoparticle comprises a superparamagnetic nanoparticle. In one embodiment, a nanoparticle comprises an iron oxide superparamagnetic nanoparticle. In one embodiment, a superparamagnetic nanoparticle does not possess magnetic properties in the absence of a magnet or a magnetic field. In one embodiment, a superparamagnetic nanoparticle possesses magnetic properties only in the presence of a magnet or a magnetic field. In one embodiment, a superparamagnetic nanoparticle possesses magnetic properties only upon being magnetized.

In one embodiment, IO is iron oxide. In one embodiment, NP is nanoparticle or nanoparticles. In one embodiment, IONP is iron oxide nanoparticle or nanoparticles. In one embodiment, SPION is superparamagnetic iron oxide nanoparticle/s. In one embodiment, IONP comprises SPION.

In one embodiment, a protein layer is an albumin layer or comprises albumin. In one embodiment, a protein layer comprises serum albumin. In one embodiment, a protein layer comprises human serum albumin.

In one embodiment, at least two layers is 4-14 layers. In one embodiment, at least two layers is 3-12 layers. In one embodiment, at least two layers is 2-7 layers. In one embodiment, at least two layers is 4-10 layers. In one embodiment, at least two layers is 3-6 layers. In one embodiment, at least two layers is 4-8 layers. In one embodiment, at least two layers is 5-9 layers. In one embodiment, at least two layers is 2-4 layers. In one embodiment, at least two layers is 3-4 layers. In one embodiment, at least two layers is 4-8 layers. In one embodiment, at least two layers is 5-7 layers.

In one embodiment, the particle is a nanoparticle. In one embodiment, the particle is a superparamagnetic particle. In one embodiment, the particle is a superparamagnetic nanoparticle.

In one embodiment, the metal chelating polymer has functional groups capable of binding metal ions selected from amino, hydroxyl, carboxylate, —SH, ether, imine, phosphate, sulfide, or any combination thereof.

In one embodiment, the polymer comprises gelatin, polymethylenimine, chitosan, polylysine or any combination thereof. In one embodiment, the metal chelating polymer is further conjugated to a fluorescent dye.

In one embodiment, the magnetic metal oxide comprises an iron oxide or a ferrite derived from an iron oxide. In one embodiment, the magnetic metal oxide comprises magnetite, maghemite, or a mixture thereof. In one embodiment, the magnetic metal oxide comprises an oxide of the formula (Fe₅M)SO₄, wherein M represents a transition metal ion, selected from: Zn²⁺, Co²⁺, Mn²⁺, and Ni²⁺.

In one embodiment, the nanoparticle has a diameter between 10 to 1000 nm. In one embodiment, the nanoparticle has a diameter between 10 to 100 nm. In one embodiment, the nanoparticle has a diameter between 30 to 300 nm. In one embodiment, the nanoparticle has a diameter between 50 to 800 nm. In one embodiment, the nanoparticle's diameter is 15 to 100 nm. In one embodiment, the nanoparticle's diameter is 15 to 90 nm. In one embodiment, the nanoparticle's diameter is 60 to 500 nm. In one embodiment, the nanoparticle's diameter is 80 to 300 nm.

In one embodiment, the median diameter of: (a) nanoparticles as described herein or (b) nanoparticles within a composition as described herein, is from 50 to 600 nm. In one embodiment, the median diameter of: (a) nanoparticles as described herein or (b) nanoparticles within a composition as described herein, is from 60 to 500 nm. In one embodiment, the median diameter of: (a) nanoparticles as described herein or (b) nanoparticles within a composition as described herein, is from 100 to 300 nm. In one embodiment, the median diameter of: (a) nanoparticles as described herein or (b) nanoparticles within a composition as described herein, is from 150 to 500 nm. In one embodiment, the median diameter of: (a) nanoparticles as described herein or (b) nanoparticles within a composition as described herein, is from15 to 150 nm.

In one embodiment, the “diameter” is the largest diameter of each particle or nanoparticle. In one embodiment, the “diameter” is the shortest diameter of each particle or nanoparticle.

In one embodiment, the magnetic metal oxide coating the aforesaid metal chelating polymer is an iron oxide or a ferrite derived from an iron oxide. In one embodiment, the magnetic metal oxide is iron oxide.

In one embodiment, a particle is synthesized in a process comprising nucleation followed by controlled growth of at least 2 layers of metal oxide onto gelatin nuclei. In one embodiment, a particle is coated by the addition of albumin to the aqueous dispersion of the NIR fluorescent particle core and shaken. In one embodiment, an active compound or agent is conjugated to the particle physically. In one embodiment, at least one active agent comprises high affinity to albumin.

In one embodiment, at least 7% w/w of the particle's weight is the at least one active agent. In one embodiment, at least 10% w/w of the particle's weight is the at least one active agent. In one embodiment, at least 15% w/w of the particle's weight is the at least one active agent. In one embodiment, at least 20% w/w of the particle's weight is the at least one active agent. In one embodiment, at least 25% w/w of the particle's weight is the at least one active agent. In one embodiment, at least 30% w/w of the particle's weight is the at least one active agent. In one embodiment, at least 35% w/w of the particle's weight is the at least one active agent.

In one embodiment, at least 37% w/w of the particle's weight is the at least one active agent. In one embodiment, at least 40% w/w of the particle's weight is the at least one active agent. In one embodiment, at least 42% w/w of the particle's weight is the at least one active agent. In one embodiment, at least 4 In one embodiment, at least 25% w/w of the particle's weight is the at least one active agent. In one embodiment, at least 47% w/w of the particle's weight is the at least one active agent. In one embodiment, at least 50% w/w of the particle's weight is the at least one active agent.

In one embodiment, the present invention surprisingly provides that physical attachment or physically conjugated at least one active agent provides a substantially higher loading of the particle or nanoparticle with at least one active agent as described herein compared to covalent binding of the at least one active agent. In one embodiment, the present invention surprisingly provides that physical attachment or physically conjugated at least one active agent results in a substantially better stability of the particle or nanoparticle as described herein compared to covalent binding of the at least one active agent.

In one embodiment, at least one active agent such as but not limited to 5-Fluorouracil and/or bevacizumab is physically conjugated and/or covalently conjugated to a particle or a nanoparticle as described herein. In one embodiment, at least one active agent such as but not limited to 5-Fluorouracil and/or bevacizumab is physical conjugated and/or covalently conjugated to a particle or a nanoparticle having a metal chelating polymer core surrounded or coated by a metal oxide such as iron oxide and having a shell or an outer shell comprising human serum albumin (HAS). In one embodiment, at least one active agent such as but not limited to 5-Fluorouracil and/or bevacizumab has high affinity to HAS.

In one embodiment, at least one active agent such as but not limited to 5-Fluorouracil and/or bevacizumab is physically conjugated to the nanoparticle (NP) in w/w ratios of 20:1 to 1:20. In one embodiment, at least one active agent such as but not limited to 5-Fluorouracil and/or bevacizumab is physically conjugated to the nanoparticle (NP) in w/w ratios of 10:1 to 1:10. In one embodiment, at least one active agent such as but not limited to 5-Fluorouracil and/or bevacizumab is physically conjugated to the nanoparticle (NP) in w/w ratios of 5:1 to 1:5. In one embodiment, at least one active agent such as but not limited to 5-Fluorouracil and/or bevacizumab (Bev) is physically conjugated to the nanoparticle (NP) in w/w ratios of 3:1 to 1:3.

In one embodiment, a nanoparticle as described herein comprises molecules of the at least one active agent physically conjugated to it and molecules of the at least one active agent, covalently conjugated to it.

In one embodiment, the at least one active agent such as but not limited to 5-Fluorouracil and/or bevacizumab is at least 20% w/w of the entire particle or nanoparticle as described herein. In one embodiment, the at least one active agent such as but not limited to 5-Fluorouracil and/or bevacizumab is at least 25% w/w of the entire particle or nanoparticle as described herein. In one embodiment, the at least one active agent such as but not limited to 5-Fluorouracil and/or bevacizumab is at least 30% w/w of the entire particle or nanoparticle as described herein. In one embodiment, the at least one active agent such as but not limited to 5-Fluorouracil and/or bevacizumab is at least 35% w/w of the entire particle or nanoparticle as described herein. In one embodiment, the at least one active agent such as but not limited to 5-Fluorouracil and/or bevacizumab is at least 40% w/w of the entire particle or nanoparticle as described herein. In one embodiment, the at least one active agent such as but not limited to 5-Fluorouracil and/or bevacizumab is at least 45% w/w of the entire particle or nanoparticle as described herein. In one embodiment, the at least one active agent such as but not limited to 5-Fluorouracil and/or bevacizumab is up to 50% w/w of the entire particle or nanoparticle as described herein. In one embodiment, the at least one active agent such as but not limited to 5-Fluorouracil and/or bevacizumab is up to 60% w/w of the entire particle or nanoparticle as described herein. In one embodiment, the at least one active agent such as but not limited to 5-Fluorouracil and/or bevacizumab is up to 70% w/w of the entire particle or nanoparticle as described herein.

In one embodiment, the at least one active agent such as but not limited to 5-Fluorouracil is 20% to 50% w/w of the entire particle or nanoparticle as described herein. In one embodiment, the at least one active agent such as but not limited to 5-Fluorouracil is 30% to 50% w/w of the entire particle or nanoparticle as described herein. In one embodiment, the at least one active agent such as but not limited to 5-Fluorouracil is 30% to 70% w/w of the entire particle or nanoparticle as described herein. In one embodiment, the at least one active agent such as but not limited to 5-Fluorouracil is 20% to 40% w/w of the entire particle or nanoparticle as described herein.

In one embodiment, at least one active agent comprises: 5-Fluorouracil and/or bevacizumab, a fluorescent dye, a contrast agent, a peptide, a peptidomimetic, a polypeptide or a small molecule. In one embodiment, at least one active agent comprises: a chemotherapeutic agent, a VEGF inhibitor, an antimetabolite drug such Fluorouracil (5-FU), a cancer drug, or any combination thereof. In one embodiment, at least one active agent comprises: Fluorouracil, bevacizumab or a combination thereof. In one embodiment, a fluorescent dye is covalently bound to the metal chelating polymer. In one embodiment, fluorescent dye includes, without being limited to, rhodamine or fluorescein. In one embodiment, the terms “bevacizumab”, “Bev” and “Avastin” are used interchangeably.

In another embodiment, an additional active agent can be entrapped or bound within the nanoparticle as described herein or covalently bound to the metal chelating polymer. Such additional active agent comprises, in one embodiment, a contrast agent, namely a compound used to improve the visibility of internal bodily structures in either an X-ray imaging or magnetic resonance imaging (MM) or a dye.

In another embodiment, at least one active agent, an additional active agent or both comprises an enzyme. In another embodiment, at least one active agent, an additional active agent or both comprises an antimetabolite drug such Fluorouracil (5-FU). In another embodiment, at least one active agent, an additional active agent or both comprises a chemotherapeutic agent. In another embodiment, at least one active agent, an additional active agent or both comprises an antibody such but not limited to a VEGF inhibitor, avastin (Bev) or remicade.

In another embodiment, at least one active agent, an additional active agent or both comprises a hormone or a polypeptide hormone such as insulin, obestatin or ghrelin.

In another embodiment, at least one active agent, an additional active agent or both comprises an anthracycline chemotherapeutic agent. In another embodiment, at least one active agent, an additional active agent or both comprises an immunotherapeutic agent. In another embodiment, at least one active agent, an additional active agent or both comprises an antimetabolite drug such as Fluorouracil (5-FU). In another embodiment, at least one active agent, an additional active agent or both comprises a cancer immunotherapeutic agent. In another embodiment, a chemotherapeutic agent comprises a VEGF inhibitor such as an antibody such as Bev. In another embodiment, a chemotherapeutic agent comprises an anti-angiogenic agent. In another embodiment, a VEGF inhibitor comprises a vascular endothelial growth factor (VEGF) inhibitor and/or a vascular endothelial growth factor receptor (VEGFR) inhibitor. In another embodiment, a VEGF inhibitor comprises an antibody directed against VEGF or VEGFR, soluble VEGFR/VEGFR hybrids, or a tyrosine kinase inhibitor. In another embodiment, a VEGF inhibitor comprises platelet-derived growth factor receptor (PDGFR) inhibitor activity. In one embodiment, VEGF inhibitor comprises bevacizumab, sunitinib, sorafenib or any combination thereof.

In another embodiment, at least one active agent, an additional active agent or both comprises daunombicin (also known as adriamycin), doxorubicin, epirubicin, idarubicin and/or mitoxantrone. In another embodiment, at least one active agent, an additional active agent or both comprises an antifolate drug. In another embodiment, at least one active agent, an additional active agent or both comprises trimethoprim, pyrimethamine and/or pemetrexed. In another embodiment, at least one active agent, an additional active agent or both comprises an antibiotic.

In another embodiment, at least one active agent, an additional active agent or both comprises an amine-derived hormone, i.e., a derivative of the amino acids tyrosine and tryptophan. Non-limiting examples of amine-derived hormones include catecholamines, e.g., epinephrine, norepinephrine and dopamine, and thyroxine.

In another embodiment, at least one active agent, an additional active agent or both comprises lipid- or phospholipid-derived hormone. In another embodiment, at least one active agent, an additional active agent or both comprises a steroidal hormone. In another embodiment, at least one active agent, an additional active agent or both comprises testosterone. In another embodiment, at least one active agent, an additional active agent or both comprises Cortisol or an eicosanoid. In another embodiment, at least one active agent, an additional active agent or both comprises an anti-inflammatory agent. In one embodiment, an anti-inflammatory agent comprises a corticosteroid or non-steroidal antiinflammatory drugs (NSAIDs) such as, but not limited to, aspirin, choline and magnesium salicylates, choline salicylate, celecoxib, diclofenac, diflunisal, etodolac, fenoprofen calcium, flurbiprofen, ibuprofen, indomethacin, ketoprofen, magnesium salicylate, meclofenamate sodium mefenamic acid, meloxicam, nabumetone, naproxen, oxaprozin, piroxicam, rofecoxib, salsalate, sodium salicylate, sulindac, tolmetin sodium and valdecoxib.

In one embodiment, the protein layer is the shell of the particle and the nanoparticle as disclosed herein. In one embodiment, the protein comprises albumin. In one embodiment, the protein comprises serum albumin.

In one embodiment, the shell's thickness is 1-20 nm. In one embodiment, the shell's thickness is 2-10 nm. In one embodiment, the shell's thickness is 3-8 nm. In one embodiment, the shell's thickness is 2-5 nm. In one embodiment, the shell's thickness is 4-10 nm. In one embodiment, the shell's thickness is 6-15 nm. In one embodiment, the shell's thickness is 2-4 nm.

In one embodiment, an additional active agent is covalently bound to the outer protein shell. In one embodiment, the nanoparticle further comprises at least one compound physically or covalently bound to the outer surface of the protein layer or shell.

In one embodiment, an additional active agent is covalently bound to the outer surface of the magnetic metal oxide is bound, via a molecule containing a functional group attached to the magnetic metal oxide surface. In some embodiments, the additional active agent comprises a polymer selected from a polysaccharide, such as but not limited to: chitosan, a protein, gelatin or albumin, a peptide, or a polyamine.

In another embodiment, the additional active agent covalently bound to the outer surface of the magnetic metal oxide is bound, in fact, via an activating ligand attached to the magnetic metal oxide outer surface. In some embodiments, the activating ligand is acryloyl chloride, divinyl sulfone (DVS), dicarbonyl imidazole ethylene glycolbis(sulfosuccinimidylsuccinate) or m-maleimidobenzoic acid N-hydroxysulfosuccinimide ester. In one embodiment, the activating ligand is DVS.

In another embodiment, the activating ligands may further be attached to the protein shell. In another embodiment, at least one active agent, an additional active agent or both is/are attached to the nanoparticle by physical binding which is based on non-covalent interactions, e.g., hydrophobic bonds, ionic interactions and hydrogen bonds, between the active agent(s) and the outer surface of the magnetic metal oxide. Thus, in another embodiment, at least one active agent, an additional active agent or both is/are physically bound to the metal chelating polymer, the metal oxide, the protein shell or any combination thereof. In another embodiment, at least one active agent, an additional active agent or both is/are attached to the nanoparticle by physical binding which are devoid of a covalent bond.

In one embodiment, the protein layer comprises a blood protein. In one embodiment, the protein layer comprises albumin. In one embodiment, the protein layer comprises serum albumin.

In one embodiment, provided herein is a pharmaceutical composition comprising a nanoparticle as described herein and a pharmaceutically acceptable carrier. The pharmaceutical composition of the present invention may be used for various biological, medical and therapeutic applications. In one embodiment, the pharmaceutical composition of the present application is used with the application of magnetic field to concentrate and drive the nanoparticle to a target tissue. In one embodiment, the pharmaceutical composition of the present invention is used for local drug delivery, drug stabilization, drug delivery and controlled release of drugs. In one embodiment, the pharmaceutical composition of the present invention comprises magnetic polymer/metal oxide composite nanoparticles as defined above.

In one embodiment, a “pharmaceutical composition” refers to a preparation of nanoparticles as described herein with other chemical components such as physiologically suitable carriers and excipients. The purpose of a pharmaceutical composition is to facilitate administration of nanoparticles to a target tissue.

In one embodiment, the phrases “physiologically acceptable carrier” and “pharmaceutically acceptable carrier” which be interchangeably used refer to a carrier or a diluent that does not cause significant irritation to an organism and does not abrogate the biological activity and properties of the administered compound. An adjuvant is included under these phrases. In one embodiment, one of the ingredients included in the pharmaceutically acceptable carrier can be for example polyethylene glycol (PEG), a biocompatible polymer with a wide range of solubility in both organic and aqueous media (Mutter et al. (1979).

In one embodiment, “excipient” refers to an inert substance added to a pharmaceutical composition to further facilitate administration of an active ingredient. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, Pa., latest edition, which is incorporated herein by reference.

In one embodiment, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In one embodiment, the preparation is administered in a local rather than systemic manner, for example, via injection of the nanoparticles directly into a specific region of a patient's body—target tissue and/or into the peritumoral tissue of a solid tumor. In one embodiment, into the peritumoral tissue is a location which is 0.1 cm to 5 cm away from a surface of the peritumoral tissue. In one embodiment, into the peritumoral tissue is devoid of intra-tumoral.

In one embodiment, administering a nanoparticle comprising a chemotherapeutic drug such as but not limited to a VEGF inhibitor such as an antibody such as Bev, is by administering the nanoparticle into the peritumoral tissue of a solid tumor. In one embodiment, inhibiting the growth of a solid tumor and reducing its volume comprises administering the nanoparticle into the peritumoral tissue of the solid tumor. In one embodiment, inhibiting the growth of a solid tumor and reducing its volume comprises applying a magnetic field to the solid tumor and administering the nanoparticle into the peritumoral tissue of the solid tumor. In one embodiment, inhibiting the growth of a solid tumor, inhibiting the area and/or the volume of vasculature surrounding the solid tumor and/or reducing the solid tumor's volume, comprises applying a magnetic field to the solid tumor and administering the nanoparticle into the peritumoral tissue of the solid tumor. In one embodiment, administering the nanoparticle into the peritumoral tissue of the solid tumor or CRC tumor is without systemically administering the nanoparticle.

In one embodiment, the present invention provides a method for reducing a dose or a dosage of a chemotherapeutic drug and inhibiting the growth of a tumor, comprising administering the chemotherapeutic drug such as but not limited to a VEGF inhibitor such as an antibody such as Bev bound, on or within a nanoparticle into the peritumoral tissue of the tumor and applying a magnetic field such as described herein. In one embodiment, a method for reducing a dose or a dosage of a chemotherapeutic drug comprises reducing toxicity. In one embodiment, a method for reducing a dose or a dosage of a chemotherapeutic drug comprises reducing liver toxicity and/or systemic toxicity. In one embodiment, reducing a dose or a dosage of a chemotherapeutic drug according to the present methods is in comparison to the effective dose or dosage of the chemotherapeutic drug in systemic administration or in administration without applying a magnetic field.

In one embodiment, the present invention provides a method for enhancing the efficacy of a dose or a dosage of a chemotherapeutic drug and inhibiting the growth of a tumor, comprising administering the chemotherapeutic drug such as but not limited to a VEGF inhibitor such as an antibody such as Bev bound, on or within a nanoparticle into the peritumoral tissue of the tumor and applying a magnetic field such as described herein. In one embodiment, the present invention provides a method for rendering an ineffective and/refractory dose or dosage of a chemotherapeutic drug effective and inhibiting the growth of a tumor, comprising administering the chemotherapeutic drug such as but not limited to a VEGF inhibitor such as an antibody such as Bev bound, on or within a nanoparticle into the peritumoral tissue of the tumor and applying a magnetic field such as described herein. In one embodiment, the present invention provides a method for prolonging the anti-tumoral effect of a chemotherapeutic drug and inhibiting the growth of a tumor, comprising administering the chemotherapeutic drug such as but not limited to a VEGF inhibitor such as an antibody such as Bev bound, on or within a nanoparticle into the peritumoral tissue of the tumor and applying a magnetic field such as described herein. In one embodiment, enhancing the efficacy of a dose or a dosage of a chemotherapeutic drug according to the present methods is in comparison to the efficacy of the dose or dosage of the chemotherapeutic drug in systemic administration or in administration without applying a magnetic field. In one embodiment, chemotherapeutic agent comprises a chemotherapeutic drug. In one embodiment, chemotherapeutic agent or a chemotherapeutic drug comprises a VEGF inhibitor or Bev. In one embodiment, chemotherapeutic agent or a chemotherapeutic drug comprises an anti-angiogenic drug. In one embodiment, chemotherapeutic agent or a chemotherapeutic drug comprises a platelet-derived growth factor receptor (PDGFR) inhibitor. In one embodiment, VEGF inhibitor comprises PDGFR inhibitor and/or PDGFR inhibitor activity. In one embodiment, chemotherapeutic agent or a chemotherapeutic drug comprise bevacizumab, sunitinib, sorafenib or any combination thereof.

In one embodiment, the present invention provides a method for reducing the risk of rendering a tumor or a solid tumor refractory and/or resistant to a chemotherapeutic agent such as a VEGF inhibitor such as Bev. In one embodiment, the present invention provides a method for reducing the risk of rendering a tumor or a solid tumor refractory and/or resistant to a chemotherapeutic agent, comprising administering the chemotherapeutic drug such as but not limited to a VEGF inhibitor such as an antibody such as Bev bound, on or within a nanoparticle into the peritumoral tissue of the tumor and applying a magnetic field such as described herein.

In one embodiment, provided herein is a method for reducing the risk of converting a bevacizumab (Bev) responsive solid tumor to a Bev non-responsive solid tumor, comprising: (a) locally administering to the Bev responsive solid tumor or to the tumor's peritumoral tissue a superparamagnetic nanoparticle comprising Bev; and (b) applying a magnetic field on the tumor; and (c) repeating steps (a) and (b) for at least 1 additional time, at least 2 additional times, at least 3 additional times, at least 4 additional times, at least 5 additional times, at least 6 additional times, at least 7 additional times, at least 8 additional times, at least 9 additional times, at least 10 additional times, at least 11 additional times, at least 12 additional times, at least 15 additional times, at least 17 additional times, at least 20 additional times, or at least 25 additional times. In one embodiment, rendering comprises converting.

In one embodiment, a method for reducing the risk of rendering a tumor or a solid tumor refractory and/or resistant to a chemotherapeutic agent comprises a method for prolonging the effective duration of a chemotherapeutic agent. In one embodiment, a method for reducing the risk of rendering a tumor or a solid tumor refractory and/or resistant to a chemotherapeutic agent comprises a method for increasing the number of effective doses of a chemotherapeutic agent or the number of treatment with the chemotherapeutic agent. In one embodiment, a method for reducing the risk of rendering a tumor or a solid tumor refractory and/or resistant to a chemotherapeutic agent comprises a method for reducing the risk of resistance to an anti-angiogenic drug or therapy.

In one embodiment, recurrent and/or rapid and/or numerous treatments of a VEGF inhibitor responsive solid tumor with an anti-angiogenic drug and/or a VEGF inhibitor such as Bev renders the solid tumor as resistant and/or refractory to treatment with an anti-angiogenic drug and/or a VEGF inhibitor such as Bev. In one embodiment, recurrent and/or rapid and/or numerous systemic treatments of an anti-angiogenic drug and/or a VEGF inhibitor responsive solid tumor with a VEGF inhibitor such as Bev administered systemically renders the solid tumor as resistant and/or refractory to treatment with an anti-angiogenic drug and/or a VEGF inhibitor such as Bev. In one embodiment, recurrent and/or rapid and/or numerous systemic treatments of an anti-angiogenic drug and/or a VEGF inhibitor responsive solid tumor with an anti-angiogenic drug and/or a VEGF inhibitor such as Bev administered locally without applying magnetic field renders the solid tumor as resistant and/or refractory to local treatment with an anti-angiogenic drug and/or a VEGF inhibitor such as Bev.

In one embodiment, the methods, systems and kits of the present invention reduce the risk of developing a tumor resistant to an anti-angiogenic drug and/or a VEGF inhibitor such as Bev, provide effective anti-angiogenic drug and/or VEGF inhibitor such as Bev treatment in a solid tumor refractory or resistant to an anti-angiogenic drug and/or VEGF inhibitor such as Bev. In one embodiment, a resistant tumor is a tumor which responses poorly to a VEGF inhibitor as determined by one of skill in the art. In one embodiment, a resistant tumor is a tumor which responses poorly to a maximal dose or a previously effective dose of a VEGF inhibitor as determined by one of skill in the art. In one embodiment, a refractory tumor is a tumor which does not respond to a maximal dose or a previously effective dose of a VEGF inhibitor as determined by one of skill in the art.

In one embodiment, local administration comprises administration to an area surrounding a target tissue or a tumor such as the peritumoral tissue. In one embodiment, local administration comprises administration in close proximity to a target tissue, to a peritumoral tissue, or a tumor.

In one embodiment, a peritumoral tissue is a peritumoral tissue of a CRC tumor. In one embodiment, a peritumoral tissue is a peritumoral tissue of a solid tumor. In one embodiment, a peritumoral tissue is a peritumoral tissue of a Bev or a VEGF inhibitor resistant tumor.

In one embodiment, inhibiting the growth of a solid tumor such as a Bev non-responsive tumor is reducing the area or volume of vasculature in and around the tumor. In one embodiment, inhibiting the growth of a solid tumor such as a Bev non-responsive tumor is maintaining or reducing tumor volume and/or weight. In one embodiment, inhibiting the growth of a solid tumor such as a Bev non-responsive tumor is maintaining or reducing tumor volume and/or weight for at least 3, 4, 5, 6, 7, 8, 9, 10, 11, 12, 13 or 14 days post treatment with a nanoparticle and magnetic field, as provided herein.

In one embodiment, a target tissue is a tissue to be scanned. In one embodiment, a target tissue is a tissue to be contacted with a nanoparticle as described herein. In one embodiment, a target tissue is a tissue to be contacted with a nanoparticle comprising at least one active agent as described herein. In one embodiment, a target tissue is a tissue to be contacted with a dye or a marker as described herein. In one embodiment, a target tissue is a tissue afflicted with a disease. In one embodiment, at least one active agent is a therapeutic agent for specifically treating the target tissue.

In one embodiment, a method as described herein comprises identifying and/or analyzing the target tissue. In one embodiment, identifying comprises: identifying location, identifying size, identifying disease type, identifying severity of the disease, identifying neighboring tissues, or any combination thereof.

In one embodiment, in a method, a system or a kit as described herein the nanoparticle may be substituted with any superparamagnetic nanoparticle known in the art. In one embodiment, a system or a kit as described herein comprises a nanoparticle as described herein and a magnetic field generator. In one embodiment, a system or a kit as described herein comprises a nanoparticle as described herein, a magnetic field generator and a nanoparticle injecting means. In one embodiment, a system or a kit as described herein comprises a nanoparticle as described herein, a magnetic field generator and a nanoparticle injecting means. In one embodiment, a system or a kit as described herein comprises a nanoparticle as described herein, a magnetic field generator and a nanoparticle injecting means to the peritumoral tissue of a solid tumor.

In one embodiment, a method, a process or a system as described herein includes a medical imaging component such as Mill and identification of the target tissue to be treated with a nanoparticle of the invention. In one embodiment, a method, a process or a system as described herein does not include a medical imaging component. In one embodiment, a method, a process or a system as described herein is based on previously obtained medical data, wherein the previous medical data includes medical identification, analysis or a combination thereof of the target tissue to be treated with a nanoparticle of the invention.

In one embodiment, the target tissue comprises a diseased tissue. In one embodiment, the target tissue comprises a solid tumor or a colorectal (CRC) tumor. In one embodiment, the target tissue comprises a solid tumor or a colorectal (CRC) tumor which is non-responsive to a chemotherapeutic drug when the chemotherapeutic drug is administered systemically and/or without the application of a magnetic field. In one embodiment, the target tissue comprises a tissue afflicted with a pathology. In one embodiment, the target tissue comprises a cancerous tissue or a malignant tissue. In one embodiment, the target tissue is a tumor. In one embodiment, the target tissue is a non-resectable tumor. In one embodiment, an active agent, an additional active agent or both is/are a therapeutic agent known for treating a malignant tissue, a cancerous tissue, a tissue afflicted with a pathology, a diseased tissue, or any combination thereof. In one embodiment, a cancerous tissue or a malignant tissue comprises a tumor. In one embodiment, the target tissue comprises a cancerous tissue or a malignant tissue comprises a solid tumor. In one embodiment, the target tissue comprises a rectal cancer or a cancerous rectum. In one embodiment, the target tissue comprises an esophageal cancer or a cancerous esophagus. In one embodiment, the target tissue comprises a pancreatic cancer or a cancerous pancreas. In one embodiment, the target tissue comprises a liver cancer or a cancerous liver. In one embodiment, the target tissue comprises a brain tumor. In one embodiment, the target tissue comprises a breast cancer tumor. In one embodiment, the target tissue comprises a single metastasis. In one embodiment, the target tissue comprises colon cancer or metastasis. In one embodiment, the target tissue comprises a cancerous prostate. In one embodiment, the target tissue comprises a tumor, a CRC tumor, a peritumoral tissue, or any combination thereof. In one embodiment, the target tissue is the tissue to be treated with a nanoparticle as described herein, the peritumoral tissue, or both.

In one embodiment, the target tissue is a non resectable tumor such as in: rectal cancer and pancreatic cancer. In one embodiment, treatment of a non resectable tumor is performed by locally administrating the nanoparticle or the pharmaceutical composition described herein. In one embodiment, the target tissue, the tumor, the CRC tumor is a non resectable tumor. In one embodiment, the target tissue, the tumor, the CRC tumor is a non resectable tumor and a tumor which is refractory or non-responsive to the chemotherapeutic agent carried by the nanoparticle such as a VEGF inhibitor such as an antibody such as Bev.

Peroral compositions, in some embodiments, comprise liquid solutions, emulsions, suspensions, and the like. In some embodiments, pharmaceutically acceptable carriers suitable for preparation of such compositions are well known in the art. In some embodiments, liquid compositions comprise from about 0.012% to about 0.933% of the desired nanoparticles, or in another embodiment, from about 0.033% to about 0.7%.

In some embodiments, compositions for use in the methods of this invention comprise solutions or emulsions, which in some embodiments are aqueous solutions or emulsions comprising a safe and effective amount of the nanoparticles of the present invention and optionally, other compounds. In some embodiments, the compositions comprise from about 0.01% to about 10.0% w/v of the nanoparticles.

In another embodiment, the pharmaceutical compositions are administered by intravenous, intra-arterial, peritumoral injection of a liquid preparation, or intramuscular injection of a liquid preparation. In some embodiments, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

In another embodiment, the pharmaceutical compositions are administered locally to a tumor surrounding tissue such as the peritumoral tissue. In another embodiment, the pharmaceutical compositions are administered locally to a blood vessel feeding the tumor, the peritumoral tissue or the target tissue.

In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

In one embodiment, pharmaceutical compositions for use in accordance with the present invention is formulated in conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

In one embodiment, injectables, of the invention are formulated in aqueous solutions. In one embodiment, injectables, of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcysteine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, pharmaceutical compositions for parenteral administration include aqueous solutions of the nanoparticles in water-soluble form. Additionally, suspensions of the nanoparticles, in some embodiments, are prepared as appropriate oily or water-based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also comprises suitable stabilizers or agents which increase the solubility of the active ingredients to allow for the preparation of highly concentrated solutions.

In another embodiment, the nanoparticles are delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In another embodiment, the pharmaceutical composition delivered in a controlled release system is formulated for intravenous infusion, implantable osmotic pump, transdermal patch, or other modes of administration. In one embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a release system can be placed in proximity to the therapeutic target, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In some embodiments, the active ingredient is in powder form for constitution with a suitable vehicle, e.g., sterile, pyrogen-free water-based solution, before use. Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the nanoparticles are contained in an amount effective to achieve the intended purpose. In some embodiments, a therapeutically effective amount means an amount of active ingredients effective to prevent, alleviate or ameliorate symptoms of disease or prolong the survival of the subject being treated.

In addition, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmellose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents(e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

In one embodiment, compositions of the present invention are presented in a pack or dispenser device, such as an FDA approved kit, which contain one or more-unit dosage forms containing the active ingredient. In one embodiment, the pack, for example, comprise metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

In one embodiment, a nanoparticle or a pharmaceutical composition may be used for X-ray imaging or magnetic resonance imaging (MRI). The present invention thus relates to a method for X-ray imaging or magnetic resonance imaging (MRI) comprising administering to an individual in need the pharmaceutical composition. The present invention thus relates to a method for X-ray imaging or magnetic resonance imaging (MRI) comprising administering to an individual in need the pharmaceutical composition and applying a magnetic field for specifically directing the nanoparticle to a target tissue.

The pharmaceutical composition of the present invention, when aimed for reducing or inhibiting the growth of a target tissue afflicted with cancer or a tumor, either with or without monitoring the size thereof, or for reducing or inhibiting the growth of cancers cells in the target tissue or tumor, are used in some embodiments with or without radiotherapy. In some embodiments, methods as described herein using a pharmaceutical composition or a nanoparticle of the invention include (i) reducing or inhibiting the growth of a cancerous tissue or a tumor; (ii) reducing or inhibiting the growth of cancerous cells within a target tissue; or (iii) reducing or inhibiting the growth of cancerous cells or a tumor and monitoring the size thereof.

The pharmaceutical composition provided by the present invention may be prepared by conventional techniques, e.g., as described in Remington: The Science and Practice of Pharmacy, 19th Ed., 1995. In one embodiment, the composition comprising a nanoparticle as described herein may be in solid, semisolid or liquid form and may further include pharmaceutically acceptable fillers, carriers or diluents, and other inert ingredients and excipients. furthermore, the pharmaceutical composition can be designed for a slow release of the nanoparticles. The composition can be administered by any suitable route, e.g. intravenously, orally, parenterally, rectally, transdermally or topically. The dosage will depend on the state of the target tissue, the magnetic field applied, the patient, and will be determined as deemed appropriate by the practitioner.

In one embodiment, the active compound and/or the additional active compound is/are bound to the particle in a manner providing a preferable release profile from the nanoparticle such as slow, immediate and/or sustained release.

The route of administration may be any route which effectively transports the nanoparticle to the appropriate or desired site of action or to a zone affected by the magnetic field.

In another embodiment, the present invention includes a method for evaluating responsiveness of cancerous cells to treatment with a candidate compound, which comprises contacting, in-vivo or ex-vivo, a target tissue with the nanoparticle or pharmaceutical composition with or without monitoring the viability of the cancerous cells, wherein the anti-cancer agent such as described herein defined as at least one active agent or an additional active agent is the candidate compound to be evaluated.

In one embodiment, at least one active compound comprise an antibody, an engineered molecule, a molecule comprising a TNF-like ligand TL1A, lymphotoxin-beta (LT-β), a CD30 ligand, a CD27 ligand, a CD40 ligand, an OX40 ligand, a 4-IBB ligand, an Apo-1 ligand, or an Apo-3 ligand, an interleukin having an anti-tumor activity selected from IL-12, IL-23 or IL-27, a cRGD peptide, a cRGD peptidomimetic, an RGD containing peptide or peptidomimetic, avastin, an anthracycline chemotherapeutic agent selected from daunorubicin, doxorubicin, epirubicin, idarubicin or mitoxantrone, an antifolate drug, or a combination thereof.

In one embodiment, at least one active compound comprises a VEGFR1 and/or VEGFR2 antibody. In one embodiment, at least one active compound comprises a VEGFR1 and/or VEGFR2 inhibitor. In one embodiment, at least one active compound comprises an anti-angiogenic agent.

In one embodiment, provided herein is a method for contacting the nanoparticle as described herein with a target tissue in a subject, comprising administering the nanoparticle or the pharmaceutical composition to the subject and applying a magnetic field, to induce contact between the target tissue and the nanoparticle.

In one embodiment, contacting the nanoparticle as described herein with a target tissue in a subject, comprising administering the nanoparticle or the pharmaceutical composition to the subject and applying a magnetic field, includes inducing contact between the target tissue and the nanoparticle.

In one embodiment, contacting comprises delivering or targeting. In one embodiment, contacting results in penetration of a nanoparticle into a cell within a target tissue. In one embodiment, contacting comprises delivering or targeting. In one embodiment, contacting results in infiltration of the nanoparticle into a cell within a target tissue. In one embodiment, the cell is a cancerous cell.

In one embodiment, contacting comprises inducing contact comprising placing the nanoparticle via a magnetic field in a distance of less than 5 mm from at least one surface of the target tissue. In one embodiment, contacting comprising inducing contact comprising placing the nanoparticle via a magnetic field in a distance of less than 5 mm from at least one surface of the target tissue. In one embodiment, contacting comprising inducing contact comprising placing the nanoparticle via a magnetic field in a distance of less than 1 mm from at least one surface of the target tissue. In one embodiment, contacting comprising inducing contact comprising placing the nanoparticle via a magnetic field in a distance of less than 0.5 mm from at least one surface of the target tissue. In one embodiment, contacting comprising inducing contact comprising placing the nanoparticle via a magnetic field in a distance of less than 0.1 mm from at least one surface of the target tissue. In one embodiment, contacting comprising inducing contact comprising placing the nanoparticle via a magnetic field in a distance of less than 50 microns from at least one surface of the target tissue.

In one embodiment, a magnetic field has magnetic field strength is in the range 0.001 to 4 Tesla. In one embodiment, a magnetic field has magnetic field strength is in the range 0.0001 to 0.1 Tesla. In one embodiment, a magnetic field has magnetic field strength is in the range 0. 1 to 4 Tesla. In one embodiment, a magnetic field has magnetic field strength is in the range 0.0001 to 0.001 Tesla. In one embodiment, a magnetic field has magnetic field strength is in the range 0.001 to 0.5 Tesla. In one embodiment, a magnetic field has magnetic field strength is in the range 0.001 to 1 Tesla.

In one embodiment, a magnetic field has a magnetic field gradient of greater than 0.2 T/m. In one embodiment, a magnetic field has a magnetic field gradient of greater than 0.5 T/m. In one embodiment, a magnetic field has a magnetic field gradient of greater than 1 T/m. In one embodiment, a magnetic field has a magnetic field gradient of greater than 2 T/m. In one embodiment, a magnetic field has a magnetic field gradient of greater than 5 T/m. In one embodiment, a magnetic field has a magnetic field gradient of greater than 10 T/m. In one embodiment, a magnetic field has a magnetic field gradient of 0.2 T/m to 20 T/m. In one embodiment, a magnetic field has a magnetic field gradient of 0.5 T/m to 30 T/m. In one embodiment, a magnetic field has a magnetic field gradient of 1 T/m to 20 T/m.

In one embodiment, a magnetic field is applied in a frequency band from 10⁸ Hz to 10²⁵ Hz. In one embodiment, a magnetic field is applied in a frequency band from 10¹⁰ Hz to 10²⁰ Hz. In one embodiment, a magnetic field is applied in a frequency band from 10¹² Hz to 10²⁸ Hz. In one embodiment, a magnetic field is applied in a frequency band from 2×10¹⁴to 10¹⁵ Hz.

In one embodiment, a magnetic field is a pulsating magnetic field. In one embodiment, a magnetic field is an oscillating magnetic field. In one embodiment, a magnetic field is a pulsating-oscillating magnetic field.

In one embodiment, a magnetic field is adjusted dynamically or statically to the target tissue. In one embodiment, a magnetic field is pre-adjusted to the target tissue. In one embodiment, a magnetic field is adapted to the target tissue. In one embodiment, adapted to the target tissue includes: adapted to the location of the target tissue, the size of the target tissue, the type of the target tissue (type of cancer or severity of a disease within the target tissue) or any combination thereof.

In one embodiment, the magnetic field is applied to the target tissue prior to administering the nanoparticle. In one embodiment, the magnetic field is applied to the target tissue during the administration of the nanoparticle. In one embodiment, the magnetic field is applied to the target tissue after the administration of the nanoparticle.

In one embodiment, magnetic field is continuously applied to the target tissue from the time prior to administration of the nanoparticle for at least one hour after the administration of the nanoparticle. In one embodiment, magnetic field is continuously applied to the target tissue from the time prior to administration of the nanoparticle to at least 2 hours after the administration of the nanoparticle. In one embodiment, magnetic field is continuously applied to the target tissue from the time prior to administration of the nanoparticle to at least 3 hours after the administration of the nanoparticle. In one embodiment, magnetic field is continuously applied to the target tissue from the time prior to administration of the nanoparticle to at least 4 hours after the administration of the nanoparticle.

In one embodiment, magnetic field is continuously applied to the target tissue to at least 30 minutes after the administration of the nanoparticle. In one embodiment, magnetic field is continuously applied to the target tissue to at least 60 minutes after the administration of the nanoparticle. In one embodiment, magnetic field is continuously applied to the target tissue to at least 90 minutes after the administration of the nanoparticle. In one embodiment, magnetic field is continuously applied to the target tissue to at least 120 minutes after the administration of the nanoparticle. In one embodiment, magnetic field is continuously applied to the target tissue to at least 180 minutes after the administration of the nanoparticle. In one embodiment, magnetic field is continuously applied to the target tissue to at least 240 minutes after the administration of the nanoparticle. In one embodiment, magnetic field is continuously applied to the target tissue to at least 300 minutes after the administration of the nanoparticle. In one embodiment, magnetic field is continuously applied to the target tissue to at least 360 minutes after the administration of the nanoparticle. In one embodiment, magnetic field is continuously applied to the target tissue to at least 420 minutes after the administration of the nanoparticle. In one embodiment, magnetic field is continuously applied to the target tissue to at least 500 minutes after the administration of the nanoparticle.

In one embodiment, magnetic field is continuously applied to the target tissue for 10 minutes and up to 800 minutes after the administration of the nanoparticle. In one embodiment, magnetic field is continuously applied to the target tissue for 20 minutes and up to 600 minutes after the administration of the nanoparticle. In one embodiment, magnetic field is continuously applied to the target tissue for 100 minutes and up to 500 minutes after the administration of the nanoparticle. In one embodiment, magnetic field is continuously applied to the target tissue for 120 minutes and up to 420 minutes after the administration of the nanoparticle. In one embodiment, magnetic field is continuously applied to the target tissue for 150 minutes and up to 300 minutes after the administration of the nanoparticle. In one embodiment, magnetic field is continuously applied to the target tissue for 180 minutes and up to 250 minutes after the administration of the nanoparticle.

In one embodiment, “administration of the nanoparticle” or “administering the nanoparticle” is done locally with the target tissue. In one embodiment, “administration of the nanoparticle” or “administering the nanoparticle” is done locally within a maximum distance of 10 cm from at least one surface of the target tissue. In one embodiment, “administration of the nanoparticle” or “administering the nanoparticle” is done locally within a maximum distance of 7 cm from at least one surface of the target tissue. In one embodiment, “administration of the nanoparticle” or “administering the nanoparticle” is done locally within a maximum distance of 5 cm from at least one surface of the target tissue. In one embodiment, “administration of the nanoparticle” or “administering the nanoparticle” is done systemically. In one embodiment, administering a particle comprises administering to the submucosa in the periphery of target tissue. In one embodiment, administering a particle comprises systemically administering.

In one embodiment, “administration of the nanoparticle” or “administering the nanoparticle” is done locally within a maximum distance of 1 cm from at least one surface of the target tissue. In one embodiment, “administration of the nanoparticle” or “administering the nanoparticle” is done locally within a distance of 0.1 mm to 15 cm from at least one surface of the target tissue. In one embodiment, “administration of the nanoparticle” or “administering the nanoparticle” is done locally within a distance of 0.5 mm to 12 cm from at least one surface of the target tissue. In one embodiment, “administration of the nanoparticle” or “administering the nanoparticle” is done locally within a distance of 1 mm to 10 cm from at least one surface of the target tissue. In one embodiment, “administration of the nanoparticle” or “administering the nanoparticle” is done locally within a distance of 1 mm to 5 cm from at least one surface of the target tissue. In one embodiment, “administration of the nanoparticle” or “administering the nanoparticle” is done locally within a distance of 4 mm to 1 cm from at least one surface of the target tissue. In one embodiment, “administration of the nanoparticle” or “administering the nanoparticle” is done locally within a distance of 0.5 mm to 8 mm from at least one surface of the target tissue.

In one embodiment, “nanoparticle” is a plurality of nanoparticles. In one embodiment, the magnetic field induces contact between al least 15% of the nanoparticles and the target tissue. In one embodiment, “nanoparticle” is a plurality of nanoparticles. In one embodiment, the magnetic field induces contact between al least 20% of the nanoparticles and the target tissue. In one embodiment, “nanoparticle” is a plurality of nanoparticles. In one embodiment, the magnetic field induces contact between al least 30% of the nanoparticles and the target tissue. In one embodiment, “nanoparticle” is a plurality of nanoparticles. In one embodiment, the magnetic field induces contact between al least 40% of the nanoparticles and the target tissue.

In one embodiment, the magnetic field induces contact between al least 50% of the nanoparticles and the target tissue. In one embodiment, the magnetic field induces contact between al least 60% of the nanoparticles and the target tissue. In one embodiment, the magnetic field induces contact between al least 70% of the nanoparticles and the target tissue. In one embodiment, the magnetic field induces contact between al least 80% of the nanoparticles and the target tissue. In one embodiment, “the nanoparticles” are the nanoparticles administered to a subject or locally to a target tissue. In one embodiment, “the nanoparticles” are the nanoparticles within a pharmaceutical composition as described herein.

In one embodiment, the magnetic field applied does not increase the temperature of the nanoparticle by more than 2° C. In one embodiment, the magnetic field applied does not increase the temperature of the nanoparticle by more than 4° C. In one embodiment, the magnetic field applied does not increase the temperature of the nanoparticle by more than 5° C. In one embodiment, the magnetic field applied does not increase the temperature of the nanoparticle by more than 7° C. In one embodiment, the magnetic field applied does not increase the temperature of the nanoparticle by more than 10° C.

In one embodiment, “at least one surface of the target tissue” is any cell within the target tissue. In one embodiment, “at least one surface of the target tissue” is any cell or surface within the target tissue. In one embodiment, “at least one surface of the target tissue” is the cell or surface within the target tissue which is closest to the location of administration of a nanoparticle or a composition comprising the same. In one embodiment, a target tissue comprises a mixture of diseased cells and normal-healthy cells. In one embodiment, a target tissue comprises a mixture of cancerous cells and normal-healthy cells. In one embodiment, a target tissue is a tumor.

In one embodiment, the term “tumor” as used herein refers to any tumor such as, without being limited to, a colorectal cancer (CRC) tumor, brain tumors, glioma, colon cancer, lung cancer, breast cancer, prostate cancer, bladder cancer, kidney cancer, ovarian cancer, pancreatic cancer, liver cancer, rectal cancer, melanoma, leukemia or multiple myeloma, glioma, and metastases thereof.

In one embodiment, provided herein is a system for delivering, concentrating and maintaining the nanoparticles as described herein in contact with the target tissue or in proximity (less than 5 cm away) to the target tissue. In one embodiment, provided herein is a system for minimizing the risk of exposure of non-target tissues to the nanoparticles by concentrating and maintaining the nanoparticles as described herein in contact with the target tissue or in proximity (less than 5 cm away) to the target tissue. In one embodiment, a nanoparticle as described herein is administered or injected into the peritumoral tissue of the tumor. In one embodiment, a nanoparticle as described herein is administered or injected into the peritumoral tissue of the tumor and maintained by magnetic field in: the peritumoral tissue, the intratumoral/tumoral tissue, or both.

In one embodiment, a magnetic field is used for delivering, concentrating and maintaining the nanoparticles as described herein. In one embodiment, an external magnetic field is applied to the target tissue or tumor so that the nanoparticles are driven and kept in close proximity or in contact to or with the target tissue or tumor or within the target tissue.

In one embodiment, a system and a method as described herein enables maintaining particles as described in proximity or within contact to or with the target tissue. In one embodiment, provided herein is a method for increasing the concentration of the particles at the vicinity of the target tissue or in contact with the with the target tissue, by administering the particle and applying a magnetic field directed to the target tissue.

In one embodiment, a system and a method as described herein enables inhibiting tumor growth in tumors that are non-responsive to Bev (wherein Bev (Avastin) is refractory in a therapeutically safe and effective amount or dose). In one embodiment, a system and a method as described herein enables inhibiting tumor growth in solid tumors or CRC tumors that are non-responsive to Bev (wherein Bev (Avastin) is refractory in a therapeutically safe and effective amount or dose). In one embodiment, diagnosing a cancer or a tumor non-responsive to Bev is known to a person of skill in the art. In one embodiment, a cancer or a tumor non-responsive to Bev is refractory to Bev. In one embodiment, a cancer or a tumor non-responsive to Bev is non-responsive or refractory to a therapeutically safe and effective amount or dose of Bev. In one embodiment, a tumor, a solid tumor, a CRC tumor or any combination thereof is/are non-responsive to Bev.

In one embodiment, a method of the invention comprises administering a nanoparticle as described herein comprising Bev to a subject in need thereof. In one embodiment, a method of the invention comprises administering a nanoparticle as described herein comprising Bev to a subject afflicted with a solid tumor, a CRC tumor or both. In one embodiment, a method of the invention comprises administering a nanoparticle as described herein comprising Bev to a subject afflicted with a solid tumor, a CRC tumor or both, wherein the solid tumor, the CRC tumor or both is/are non-responsive to Bev.

In one embodiment, a method of the invention comprises locally administering to a tumor, a nanoparticle as described herein comprising Bev. In one embodiment, a method of the invention comprises locally administering to a tumor non-responsive to Bev, a nanoparticle as described herein comprising Bev. In one embodiment, a method of the invention comprises locally administering to a tumor non-responsive to Bev, a nanoparticle as described herein comprising Bev and applying a magnetic field as described herein. In one embodiment, a method of the invention comprises locally administering to a tumor non-responsive to Bev, a nanoparticle as described herein comprising Bev and applying a magnetic field on the tumor, as described herein. In one embodiment, a method of the invention comprises locally administering to a tumor non-responsive to Bev, a nanoparticle as described herein comprising Bev and applying a magnetic field for 0.5 to 12 hours, on the tumor, as described herein. In one embodiment, 0.5 to 12 hours is: 0.5 to 10 hours, 0.5 to 8 hours , 1-6 hours, 2-6 hours, 2-5 hours, 1-3 hours, 0.5-4 hours, 3-6 hours, 3-5 hours, 3 to 10 hours, 2 to 12 hours, or 4 to 8 hours.

In one embodiment, a method of the invention comprises diagnosing a tumor as a Bev non-responsive tumor. In one embodiment, a method of the invention comprises: (a) diagnosing a tumor in a subject afflicted with cancer as a Bev non-responsive tumor; (b) locally administering to the tumor a nanoparticle comprising Bev; and (c) applying a magnetic field on the tumor.

In one embodiment, a method of the invention comprises converting a non-effective dose of Bev to an effective Bev dose, comprising: (a) locally administering to the tumor a nanoparticle comprising Bev; and (b) applying a magnetic field on the tumor. In one embodiment, a method of the invention comprises converting a non-effective dose of Bev to an effective Bev dose, comprising: (a) diagnosing a tumor in a subject afflicted with cancer as a Bev non-responsive tumor; (b) locally administering to the tumor a nanoparticle comprising Bev; and (c) applying a magnetic field on the tumor.

In one embodiment, “locally administering”, “locally administering to a tumor”, “locally administering to a solid tumor” is administering into the peritumoral tissue of the tumor. In one embodiment, a tumor or cancer is a solid tumor, a CRC tumor a BEV non-responsive tumor or any combination thereof. In one embodiment, peritumoral tissue comprises peritumoral stroma. In one embodiment, peritumoral tissue comprises aberrant angiogenesis.

In one embodiment, provided herein is a method for decreasing the total amount of chemotherapy carried by the particles, comprising administering the particle at the vicinity of the target tissue and applying a magnetic field directed to the target tissue. In one embodiment, provided herein is a method for decreasing the total number of particles, comprising administering the particles at the vicinity of the target tissue and applying a magnetic field directed to the target tissue. In one embodiment, a magnetic field comprises a personalized magnetic field. In one embodiment, personalized magnetic field is a magnetic field specifically adapted to a patient, a disease or a target tissue. In one embodiment, provided herein is a method for increasing the total amount of particles contacting a target tissue, comprising administering the particle at the vicinity of the target tissue and applying a magnetic field directed to the target tissue. In one embodiment, applying a magnetic field is before administering the particle, concomitantly with particle administration, 60 second to 500 minutes after administration of the particle, or any combination thereof.

In one embodiment, administering a particle comprises using EUS guided needles with or without a specifically designed belt of magnets focusing the magnetic fields to the target tissue.

In one embodiment, the invention further provides a system comprising the particle and a magnetic field generator. In one embodiment, the invention further provides a system comprising a magnetic field generator and a computer implemented software. In one embodiment, the software is adapted to optimize the applied magnetic field. In one embodiment, the software is adapted to optimize the magnetic field's: shape, intensity, dispersion, etc., based, for example, on the type of target tissue, tumor location, etc.

In one embodiment, the software is adapted to receive data concerning the target tissue from Mill and adapt the magnetic field to provide optimal coverage to the target tissue, such as inducing maximal contact between particles and target tissue.

In one embodiment, suitable for the purposes of the invention “magnetic field” refers to a signal generated by a magnet as the source of magnetic field, the shape and field strength to be capable of attracting magnetic particles of the invention together with agents) against the other acting on them physical phenomena.

In one embodiment a magnet is an external magnet-placed externally with respect to the treated subject. In one embodiment a magnet is an internal magnet-placed internally within the treated subject.

In one embodiment a magnet or an array of magnets is/are contained within a container or a pouch. In one embodiment a magnet or an array of magnets is/are contained within a container or a pouch such as a wearable item placing the magnet or magnets in proximity to the target tissue. In one embodiment a container or a pouch comprises a vest, a bra or any other wearable item. In one embodiment a container or a wearable comprises a helmet. In one embodiment a magnet or an array of magnets is/are insertable into a body cavity. In one embodiment a magnet or an array of magnets is/are insertable into a body location.

Suitable magnetic fields for the invention in vivo applications should preferably have a field strength of at least 50 mT (milli-Tesla), at least 100 mT, at least 200 mT, at least 300 mT, at least 400 mT, at least 500 mT, at least 1T (Tesla) or more. In one embodiment, the magnetic field comprises a magnetic field gradient greater than 0.1 T/m, greater than 0.5 T/m, greater than 1 T/m, greater than 10 T/m, greater than 25 T/m.

A “magnet” for the purposes of the present invention, comprises any suitable magnet. In one embodiment, a magnet as described herein comprises a permanent magnet. In one embodiment, a magnet as described herein comprises an electromagnet. In one embodiment, a magnet comprises a control for controlling the intensity (the field strength of the magnet) via appropriate measuring and control instruments, which are connected to the magnet.

In one embodiment, the magnetic field as used herein provides multiple concurrent magnetic fields or magnetic field/s intensity/ies. In one embodiment, the center of the target tissue is targeted with the highest intensity wherein the surroundings of the target tissue within 10 cm, 7 cm, 5 cm, 1 cm, 0.5 cm, 1 mm, 0.5 mm from the center of the target tissue receive lower magnetic field intensity. In one embodiment, the center of the target tissue is targeted with the lowest intensity wherein the surroundings of the target tissue within 10 cm, 7 cm or 5 cm, 1 cm, 0.5 cm, 1 mm, 0.5 mm from the center of the target tissue receive the highest magnetic field intensity. In one embodiment, highest magnetic field intensity is at least 5, 10, 15, 25, or 100 times higher compared to the lowest intensity.

In one embodiment, the magnet or the magnetic field is/are external. In one embodiment, externally applied magnetic field is permanent.

In one embodiment, the externally applied magnetic field is not permanently present, preferably only a part of the period of treatment as described herein. In one embodiment, the magnetic field is active only during and after administration of the particle at the proximity of the target tissue.

In one embodiment, the externally applied magnetic field is a pulsating magnetic field. In one embodiment, the maximum desired field strength is preferably achieved at the maximum of the pulse, while the minimum of the pulse preferably as low as possible, more preferably less than 20%, even more preferably less than 10% field strength of the previously applied field strength, and even more preferably bears no field strength.

In one embodiment, a pulsating magnetic field is generated with the use of an electromagnet with direct current or with alternating current. In one embodiment, a particle is administered via a directional transport. In one embodiment, directional transport according to the methods and systems as described herein comprise the driving of the particles by an external magnet (outside of the animal to be treated (mammalian, or human). The directional transport in defined regions of the target tissue.

In one embodiment, the magnet is a moveable magnet or a positional magnet (can be moved or re-positioned) free to move. Freely movable means, for example. That the magnet is guided manually or automatically for driving the particles to the target tissue. The magnet and/or the magnetic field is guided and/or determined in some embodiments, electronically or via computer-controlled variable, such as pivotable, adjustable, lockable, or any combination thereof.

In one embodiment, the magnet comprises a plurality of magnets such as an arrangement of magnets or electromagnets that can be activated either sequentially or simultaneously. In one embodiment, the magnet is set to provide a certain field strength or an oscillating and/or pulsed applied magnetic field, as described above.

In another embodiment, a magnetic field generation means provides focused magnetic particle therapy. The second control means is adapted to receive planning data for planning treatment of the subject, and the therapeutic system is adapted for performing therapy using the planning data. The second and first control means can be implemented using a single control means. The second control means can be implemented using a computer, a microcontroller, a microprocessor, an array of microprocessors, a digital electronic circuit, and an analog electronic circuit. The second control means and/or the first control means can comprise a computer program product.

In an embodiment, the magnetic field generation means comprises a magnet. In various embodiments, the magnet can comprise a superconducting magnet, a permanent magnet, an electromagnet, and/or coils for generating a magnetic field. The electromagnet can ideally be turned off when magnetic resonance imaging data is not being acquired to make it easier to generate the low or zero magnetic field region necessary to perform magnetic particle imaging and/or focused magnetic particle imaging.

In another embodiment, the magnetic field generation means is further adapted for acquiring medical image data within an imaging zone using magnetic particle imaging. The imaging zone comprises the first region and the second control means are adapted for generating planning data using the medical image data.

In another embodiment, the invention includes magnetic particle imaging which allows the very precise determination of the quantitative local distribution of magnetic nanoparticles within a subject. The knowledge of the quantitative local distribution of the magnetic nanoparticles relative to the anatomy of the subject is useful in planning therapy. Anatomical data can be acquired using an MRI scanner, a positron emission tomography scanner, a Single Photon Emission Computed Tomography scanner, a 3D X-Ray imaging system, a 3D ultrasound imaging system or a computer tomography scanner and then compared with the medical image data obtained from the magnetic particle imaging.

In another embodiment, the system further comprises a magnetic resonance imaging system adapted for acquiring medical image data within an imaging zone. The imaging zone comprises the first region and the second control means is adapted for generating planning data using medical image data. This embodiment is advantageous, because magnetic resonance imaging data contains useful anatomical information for planning the treatment of a subject. Magnetic resonance imaging gives very detailed anatomical information and magnetic particle imaging gives very detailed information on a local distribution of magnetic nanoparticles within a subject. These two imaging modalities are therefore very complimentary for planning the treatment of a subject.

In another embodiment, a system as described herein further comprises a magnetic resonance imaging system. In another embodiment, a system as described herein is adapted for acquiring medical image data. The therapeutic system is adapted for identifying the location of a target tissue within the subject using the medical image data. The target tissue, in some embodiments, can be identified using well-known image segmentation techniques.

In another embodiment, a system as described herein is adapted for generating real time planning data using the location and shape of the target tissue. In another embodiment, generating real time planning data is done by target tissue shape and deformation models implemented in software. Such models, in some embodiments are trained.

In another embodiment, the computer program product and methods utilizing the same further comprises the steps of: receiving planning data for planning treatment of the subject, controlling the treatment of the subject using the planning data, and controlling the location of the particles using the magnetic field generation means. [0051

In another embodiment, the computer program product further comprises the steps of: acquiring medical image data within an imaging zone using magnetic particle imaging and/or magnetic resonance imaging and generating planning data using the medical image data. The imaging zone comprises the target tissue.

In another embodiment, the computer program product further comprises the steps of acquiring medical image data at periodic intervals, identifying the location and shape of a target tissue within a subject using the medical image data acquired at periodic intervals, generating real time planning data using the location and shape of the target tissue, and adjusting location of the particles based upon motion and/or deformation of the target tissue using the real time planning data.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Cell Culture

CT26 cells were cultured in growth media containing high glucose DMEM (Biological Industries, 01-055-1A), 10% FBS (Biological Industries, 04-127-1A), 1% L-glutamine (Biological Industries, 03-031-1B), 1% Penicillin-streptomycin (Biological Industries, 03-033-1B). The cell culture was maintained at 37° C., 5% CO₂ in a humid chamber incubator, and was allowed to grow up to 70% confluence. Sub-culturing was performed at 70% confluence using trypsin (0.25%)-EDTA (0.02%) (Biological Industries, 03-050-1A).

Male BALB/c mice (6 weeks of age, Envigo, Israel) were used for all in-vivo experiments. All experiments were performed with approval and in accordance with the guidelines of the Institutional Animal Care and Use Committee at The Hebrew University of Jerusalem. 1.5×10⁶ CT26 cells were injected subcutaneously into the right anterior limb of each mouse. Ten days later (day 0) colorectal tumor appeared at the injection site. The mice were randomly allocated to control and experimental groups.

### In-Vivo NP Infiltration Assays

4×IONP-HSA or 6×IONP-5FU were injected either systemically or locally in tumor-bearing mice. An external magnetic field was applied for different periods of time (2,4,24 hours. Systemic injection of 4×IONP-HSA was administered through the tail vein; Local administration was performed by injection of 4×IONP-HSA or 6×IONP-5FU under the tumor. Magnetic field was applied by suturing an N35 neodymium magnet embedded in a plastic chassis (experiment) or the plastic chassis without a magnet (control) on top of the tumor for 2,4 or 6 hours. Following the injection and magnetic field application the mice were sacrificed and the tumors, spleens, and livers were excised for histological analyses.

### Anti-Tumor Activity of Systemically Administered 5FU in Tumor-Xenografted Mice

Following tumor creation systemic administration via intraperitoneal (IP) injection of 5FU (experiment) or normal saline (control) was performed. 5FU injections were carried out on days 3, 4, 5. Tumor progression was evaluated by measuring the tumor volume using a Vernier caliper at days 0,1,3,4,6,8 and 10.

### Anti-Tumor Activity of 6×IONP-5FU or 5FU in Tumor-Xenografted Mice

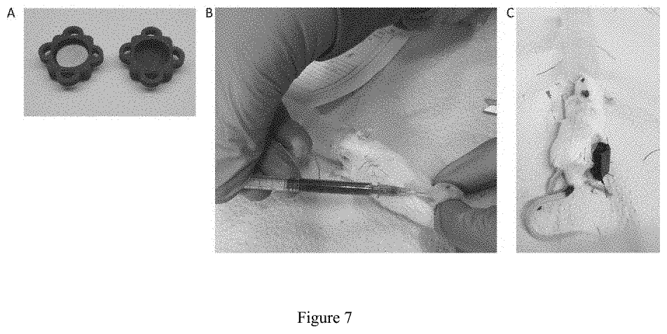

Ten days post-injection of CT26 cells (day 0) the mice were randomly divided into four groups:
1) 6×IONP-5FU exposed to 4 hours magnetic field (experimental 1)
2) 6×IONP-5FU not exposed to a magnetic field (control 1)
3) 6×IONP (with no chemotherapy) exposed to 4 hours magnet (control 2). The mice were anesthetized, tumor size was measured, and the tumor volumes were calculated. The 6×IONP-5FU or 6×IONP (with no 5FU) were injected locally underneath the tumors, and the magnet or its plastic chassis was sutured to the skin above the tumor as described in 2.1 (FIG. 7). four hours after application of the magnets or chassis only (in the control group) they were removed. The tumor volumes were measured at days 3,4,5,6,7. A second injection of 6×IONP-5FU or 6×IONP and magnet application were performed on day five. On day seven the mice were euthanized, and the tumors, spleens, and livers were extracted for histological analysis.

### Histological Analyses

The spleen liver and tumor were fixed in 4% formalin followed by dehydration and embedding in paraffin. Paraffin blocks were sectioned at approximately 3 μm-5 μm thickness. The tissue sections were stained with Hematoxylin & Eosin (H&E) and Prussian blue (PB, iron stain) according to standard protocol. Embedding, sectioning and stained slides preparation was performed by CDX Diagnostics, Jerusalem, Israel. The slides were examined by Axioskop (Zeiss, Göttingen, Germany). Tumor sections pictures were taken at ×5 and ×40 magnification. The ×5 magnification pictures were stitched using Photoshop CC software (Adobe, version 20.0.1). For iron staining quantification in the spleen 15-19 pictures of each sample at x40 magnification were taken. The pictures were segmented using Trainable Weka Segmentation plugin of ImageJ software (NIH, version 1.52i), and the blue stained area was measured and divided by the tissue area.

### Statistical Analyses

Statistical analyses of tumor volumes were performed using RM two-way Analysis of Variation (ANOVA) with a confidence interval of 95% using post hoc Dunnet' s analysis of multiple comparisons for the tumor treated with 5FU bounded to 6×IONPs and Sidak's analysis for the tumor treated with free 5FU. Statistical analysis of iron staining quantification in the spleen was performed using the nonparametric Kruskal-Wallis test with a confidence interval of 95% using post hoc Dunn's analysis of multiple comparisons.

### Example 1

### Synthesis of NIR Fluorescent iron Oxide Nanoparticles (FIONP)

The synthesis of iron oxide (IO) nanoparticles is performed by nucleation followed by controlled growth of (IO) layers onto gelatin nuclei.

Procedure: A glass bottle containing 72 ml of ddH₂O and 0.72 g of porcine gelatin was shaken at 60° C. for 30 min. The pH of the gelatin solution was then increased to 8.5 with NaOH aqueous solution (1N). Then, 6.52 mg of Cy-7 dissolved in 2 ml of dimethyl sulfoxide were added dropwise to the shaken gelatin solution. After 2 h, additional 168 ml of water were added, following by adding a few drops of 1N HCl aqueous solution to reach pH-5.5.

IO (iron oxide) NPs first coating (X1) were then prepared by adding 480 μl of FeCl₂ solution (16 mmol/5 ml 0.1N HCl) to the former 240 ml shaken fluorescent gelatin aqueous solution at 60° C., followed by the addition of NaNO₃ solution (6 mmol/5 ml H₂O). 1N NaOH aqueous solution was then added up to pH 9.5. This procedure was repeated three-6 times with 10 min intervals. After completion of the coatings the IO dispersion was allowed cooled down to room temperature. The formed magnetic NPs were then washed from excess reagents using high gradient magnetic field (HGMF) technique. For this purpose, magnetic columns containing steel fibers were used. The NPs were washed by passing the magnetic NPs aqueous dispersion through the column, under a static magnetic field and collected inside the magnetic column. An aqueous washing solution was added to the magnetic column in order to remove undesired excess reagents. The magnetic field was removed from the column and the NPs were eluted by adding an aqueous bicarbonate buffer (BB, 0.1M, pH=8.3) or PBS. The obtained IO core nanoparticles dispersed in an aqueous solution were composed of gelatin nuclei surrounded by IO layers and a very thin gelatin coating protecting the IO from agglutination in the aqueous continuous phase. For sterilization, it is common to pass the IO aqueous dispersion through 0.2 μm filter.

Concentration of the IO NPs was determined by UV (990 μl saline and 10 μl of nanoparticles).

### Example 2

### Human Serum Albumin (HSA) Coating onto the Fluorescent IO NPs

HSA coating on the IO NPs was performed by the addition of 20% (w/w) HSA (MW ˜66,000) to the aqueous dispersion of the NIR fluorescent IO core NPs and shaken at 75° C. for 12 h. The formed fluorescent IO/HSA core/shell NPs were then cooled down to room temperature and then washed with PBS (pH 7.4) using magnetic columns.

### Example 3

### Physical Conjugation of 5-Fluorouracil onto the IO/HSA NPs

The physical conjugation of 5-Fluorouracil (5FU) to the fluorescent IO/HSA NPs was based on the high affinity of the drug to HSA. In a typical procedure, 5-fluorouracil was bound to the IO/HSA NPs by adding 100 mg of the 5FU to 10 ml of the IO/HSA nanoparticles dispersed in PBS (10.0 mg/ml). A weight ratio of 1:1 IO/HSA: 5FU was maintained for the reaction. The reaction mixture was shaken at room temperature for 12 h. The resultant 5FU-physically conjugated nanoparticles were washed off excess unbound 5FU by a magnetic column with PBS (0.01M, pH 7.3).

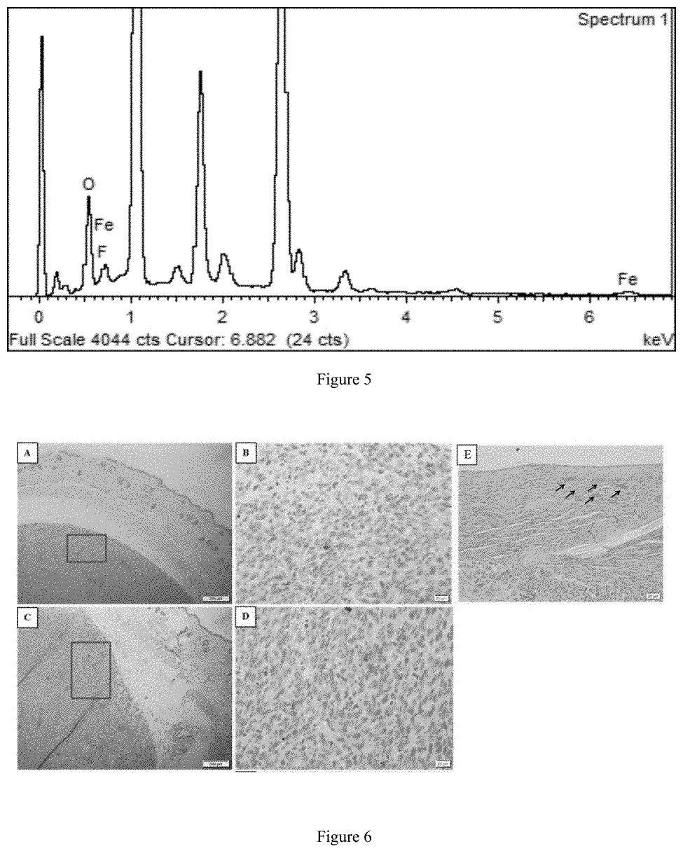

The bound/unbound concentrations of 5FU was determined through a calibration curve by UV measurements. The calculated bound 5FU concentration was 0.42 mg/1 mg of the IO/HSA nanoparticles (FIG. 5 and table 1).

TABLE 1

| Element | Weight % | Atomic % |
|---|---|---|
| O K | 61.57 | 73.29 |
| F K | 20.56 | 20.61 |
| Fe K | 17.87 | 6.09 |
| Totals | 100.00 | |

### Example 4

### Covalent/Physical Conjugation of AVASTIN onto IO/HSA NPs

### Covalent Binding of AVASTIN (AV) to IO/HSA

200 μl of NHS-PEG-MA [MA=melimide] (MW=3000 g/mol) were added to sterile IO/HSA nanoparticles dispersion in PBS (5 ml, 10 mg/mL). The reaction mixture was shaken at room temperature for 30 min. The derivatized PEGMA-conjugated IO/HSA NPs PBS dispersion was washed and eluted with bicarbonate buffer in a magnetic column, followed by immediate addition under sterile conditions of 40 μl of AV aqueous solution (25 mg/ml). The reaction mixture was shaken at 4° C. for 1.5 h. then, 10 weight % glycine solution (10 mg) were added to the AV-conjugated IO/HSA NPs bicarbonate aqueous dispersion to block residual sites. The AV-IO/HSA nanoparticles were then washed and eluted through a magnetic column with PBS and stored under sterile conditions.

Similar procedure was accomplished for the preparation of the control fluorescent Glycine-conjugated IO/HSA NPs substituting the AV glycine.

### Physical Binding of AVASTIN to IO/HSA Particles

Avastin was physically conjugated to the IO/HSA nanoparticles by adding 7.5 mg of avastin to 4 mL of the IO/HSA NPs PBS dispersion (10 mg/mL). A weight ratio of 1:5, IO/HSA:Avastin was maintained and the reaction mixture was shaken at room temperature for 12 h. The obtained avastin-physically conjugated IO/HSA NPs were washed on a magnetic column with PBS (0.01 M, pH 7.3) and collected the supernatant to determine the unbound concentrations of Avastin.

The attached and remaining concentrations of physically and covalently conjugated avastin was determined through ELISA assay using a standard protocol. The calculated covalent bound avastin concentration (via ELISA method) was 0.10 mg/1 mg of the nanoparticles. The calculated physical conjugated avastin concentration (via ELISA method) was 0.13 mg/1 mg of the nanoparticles (30% more of the covalent binding process).

### Example 5

### Cell Uptake (FACS) of IO NPs by SW620 and A172 Lines

Cell uptake analyzed by flow cytometry. SW620 or A172 cells (3×10⁵) were treated with IO NPs 0.25 mg/ml for 6 h. After incubation, cells were trypsinized, washed with culture medium, counted and suspended in PBS. The cell suspension was analyzed by flow cytometry BD FACSAria™ III (BD Biosciences, San Jose, Calif., USA) with 633 nm laser. A minimum of 10,000 cells were analyzed for each histogram generated. Gate SSC/FSC was used to exclude fragments and aggregates from the cell count. Untreated cells were used as control. Results were analyzed using FlowJo software.

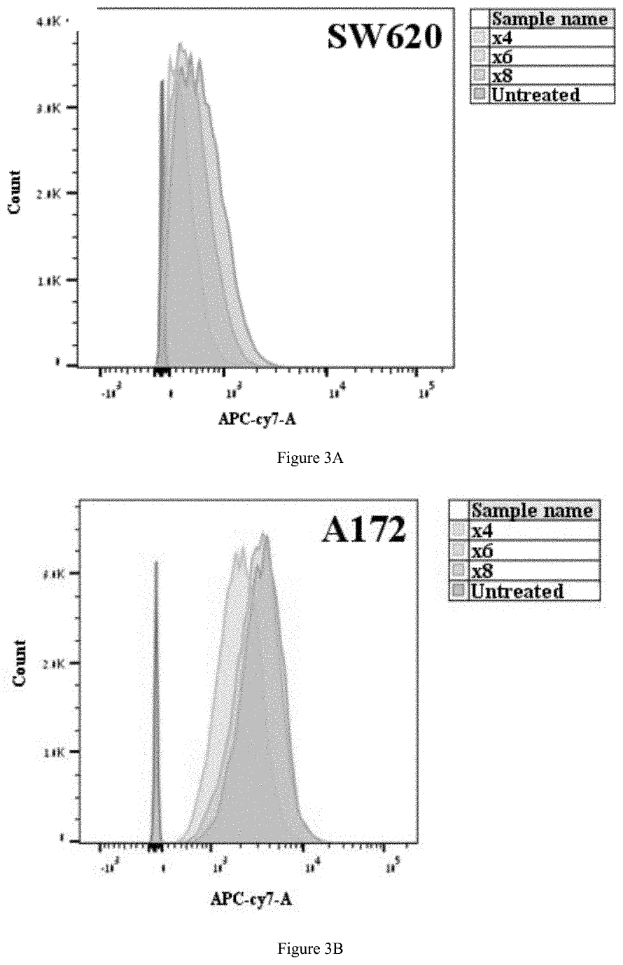

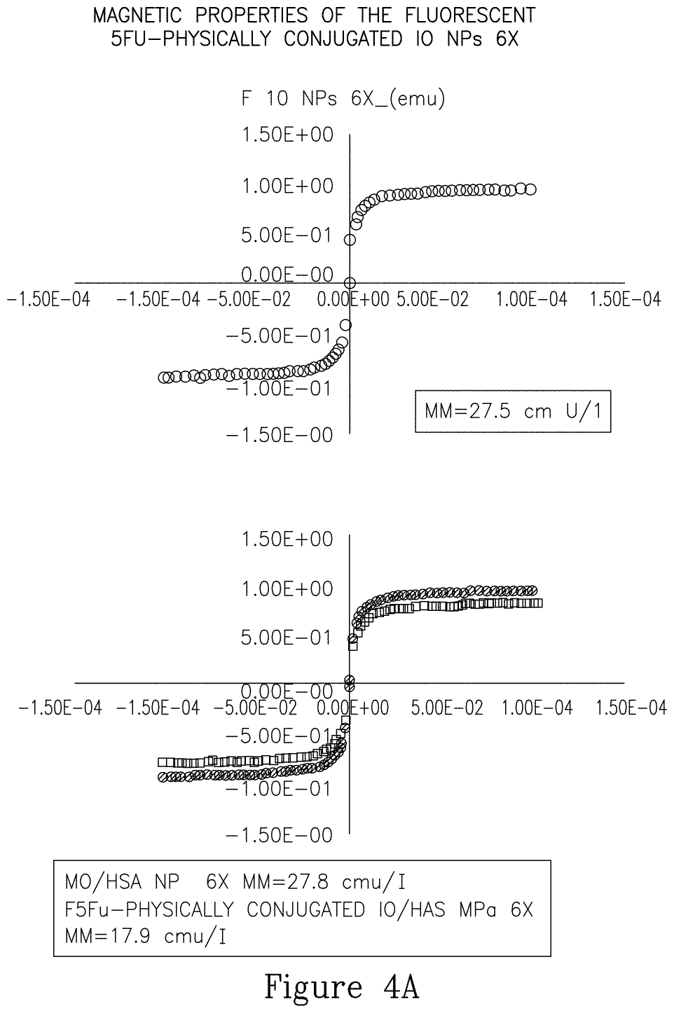

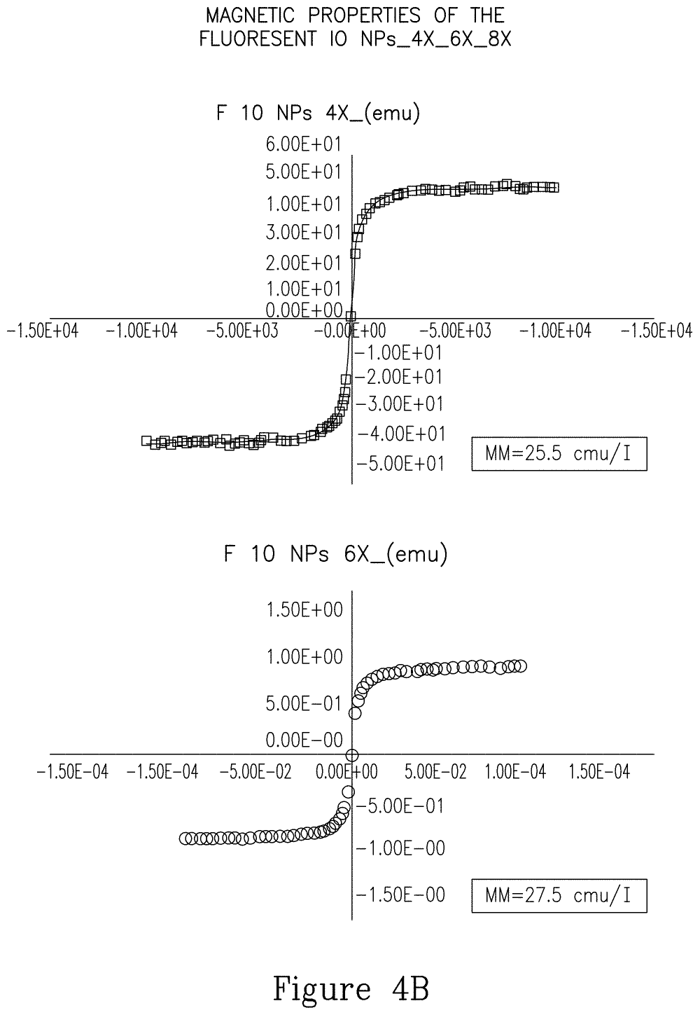

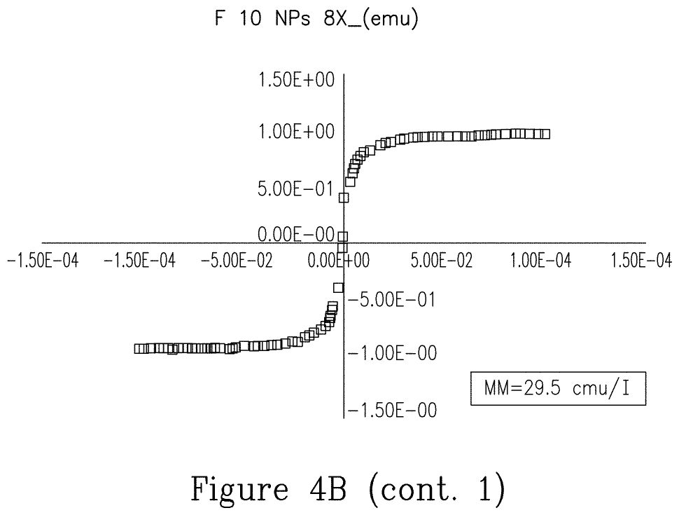

Using flow cytometry, cell uptake of 4 iron oxide layer-coated. 6 iron oxide layer—coated and 8 iron oxide layer-coated (4×, 6× and 8×) IO NPs were studied. FIGS. 3(a) (b) below exhibit the intracellular fluorescence of Cy7 as a kind of IO NPs (4×, 6×, 8×). Cells treated with 8× (iron oxide layers) IO NPs show higher progressive shift in the fluorescence relative to the untreated cells whereas the cells treated with 4× (iron oxide layers) IO NPs exhibited a less progressive shift. The progressive shifts conclude that the 8 (iron oxide layers)×IO NPs exhibit maximal and 4×IO NPs exhibit minimal cell uptake.

### Example 6

### Cytotoxicity of Non-Conjugated and Avastin-Conjugated (Physically/Covalently) IO/HSA NPs on HUVEC (Human Umbilical Vein Endothelial) Cells

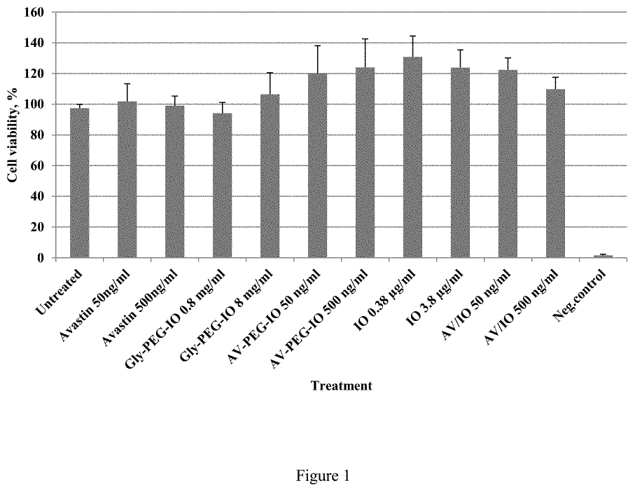

XTT assay was used in order to assess cell viability. The different IO/HSA NPs were freshly dispersed in PBS and then added to the 95% confluent cell culture in culture medium so that the final concentration of avastin-IO/HAS NPs was between 0.5 and 0.05 μg/mL. The cell cultures were further incubated at 37° C. in a humidified 5% CO₂ incubator and then checked for cellular viability after 48 h. The percentage of cell viability was calculated as shown in the manufacturer's protocol of the XTT toxicity detection kit. All samples were tested in six folds. The results are shown below in FIG. 1. The XTT results in FIG. 1 exhibit that the viability of the HUVEC cells was unharmed for 48 h of treatment with the different IO NPs.

### Example 7

### Cytotoxicity of Physically-Conjugated 5FU-IO/HSA NPs on Human Colon Adenocarcinoma (SW620) and Human Neuroblastoma (A172) Cell Line

The activity of 4-coated physically-conjugated 5FU-IO/HSA NPs (4× 5-FU) and 4× (iron oxide layers), 6× (iron oxide layers) and 8× (iron oxide layers) control NPs were examined with SW620 and A172 cell lines (5×10³/cell).

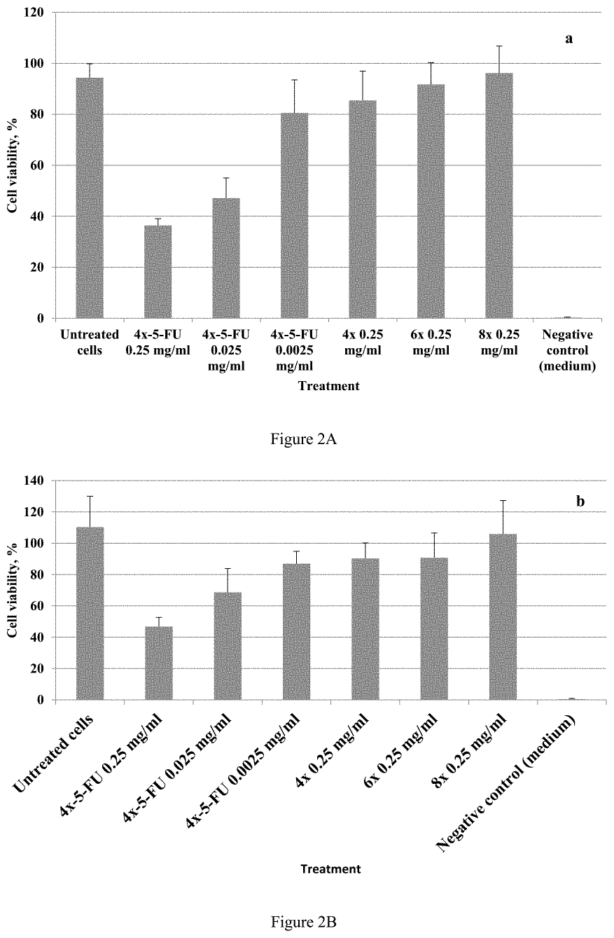

FIGS. 2(a) and (b) summarizes the results of these trials and shows that the control NPs (IO/HSA NPs) do not possess significant toxicity to both cell lines. On the other hand, both concentrations of physically-conjugated 5FU-IO/HSA NPs (0.25 and 0.025 mg/ml) demonstrate significantly reduce in cell viability level. Minimal concentration of physically-conjugated 5FU IO/HSA NPs (0.0025 mg/ml) demonstrates only low-level cytotoxicity that resembles to control NPs. In addition, FIG. 2(a) (b) shows that the control NPs (4×, 6× and 8×) did not show any significant toxicity to both cells.

### Example 8

### Magnetic Behavior of IO NPs, IO/HSA NPs and 5FU-Physically Conjugated IO/HSA NPs

The magnetic moment of the IO NPs, as expected, is decreasing as long as the number of IO coatings is increasing (FIG. 5)

The magnetic moment observed of 6× (iron oxide layers) IO NPs and IO/HSA NPs is 27.8 emu/g whereas of 5FU-physically conjugated IO/HAS is 17.9 emu/g. The magnetic behavior show that the IO NPs retains their superparamagnetic behavior even after physically conjugated with 5 FU.

### Example 9

### Diameter of the Core (IO) and Core/Shell IO/HSA NPs

HRTEM and HRSEM measurements indicated that the diameter of the NIR fluorescent IO 4× (4 iron oxide layers) NPs and the IO/HSA 4× (4 iron oxide layers) NPs prepared at 75° C. as described in the previous part were 16±2 nm and 20±3 nm, respectively. These results indicate that the average dry diameter of the HSA coating was approximately 4.0 nm. On the other hand, DLS measurements indicated that the hydrodynamic diameter of these NIR fluorescent IO 4× (4 iron oxide layers) NPs and the IO/HSA 4× (4 iron oxide layers) NPs were 103±14 nm and 43±5 nm, respectively.

These diameter differences between TEM and DLS measurements can be attributed to the fact that TEM measures the dry diameter while DLS determines the hydrodynamic diameter, which takes into account the hydrated layers on the particle surface. The core IO NPs are composed of gelatin and IO nuclei, IO layers and a thin layer of surface gelatin which protects these core NPs from agglomeration. Gelatin is known for its ability to swell by absorbing water molecules up to 10 times of its original size. These water absorbing layers may be the main reason for the significant size difference between the dry and the water dispersed IO NPs (16±2 and 103±14 nm, respectively). It may also be postulated that the coating of the IO core NPs with HSA, through denaturation and precipitation of the HSA onto the IO core NPs partially, prevents water molecules from interacting with the gelatin belonging to the core NPs, resulting in a decreased hydrodynamic diameter, from 103±14 nm to 43±5 nm.

In addition, HRTEM measurements indicated that the diameter of the NIR fluorescent IO 4× NPs (4 iron oxide layers) was 16±2 nm and that of IO 6×4 (6 iron oxide layers) NPs was 24±3 nm. This indicates that 2 more coating layers of IO led to increased diameter of 6 nm. This may indicate that each layer of IO coating adds about 3 nm increased diameter. HRTEM of the fluorescent IO 6× NPs, indicating that the dry diameter of these NPS is 24±3 nm. HRTEM of the fluorescent IO/HSA 6× NPs, indicated that the thickness of the HSA coating is about 3-4 nm.

### Example 10

### Targeting IONP into CT26 Tumors using Magnetic Field Exposure

4×IONP-HSA were injected to the tail vein of tumor-bearing mice, and an external magnetic field was applied on top of the tumor for 2, 4, and 24 hours. Iron staining of the tumors excised from mice treated by systemic administration of 4×IONP-HSA and exposure to an external magnetic field for 24 hours, showed very weak staining in only one out of three mice (FIG. 6E). In that mouse blue staining was found only at the skin above the tumor. In all control mice which not exposed to a magnetic field, no staining for iron was visible. In two out of seven tumors exposed to 4 hours of the external magnetic field, a weak iron staining was found in dorsal areas of the tumor, in proximity to the skin. Stronger staining was found only in one out of seven tumors also in the dorsal areas (FIG. 6A-D).

These results suggest the nanoparticles infiltrated into the tumor but with low efficacy. Magnetic field exposure for 2 hours yielded no iron staining in all of the seven mice.

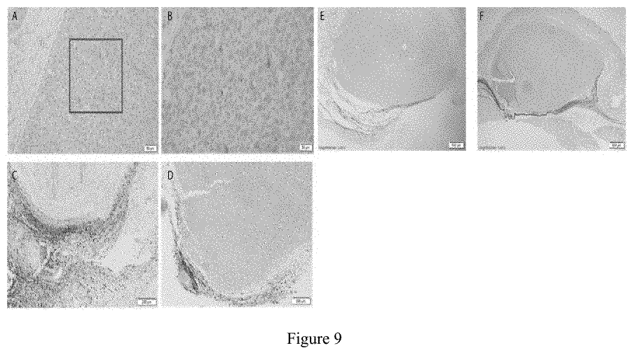

With respect to the findings arisen by systemic administration of the nanoparticles, the hypothesis that local administration of the drug-loaded nanoparticles will be more efficient in targeting the particles into the tumor while inhibiting their clearance was raised. Local administration of 4×IONP-HSA directly underneath the tumor and exposure to a magnetic field for 2 hours showed higher staining for iron surrounding the tumor and some staining in intra-tumoral regions as opposed to control with no staining inside the tumor (FIG. 9).

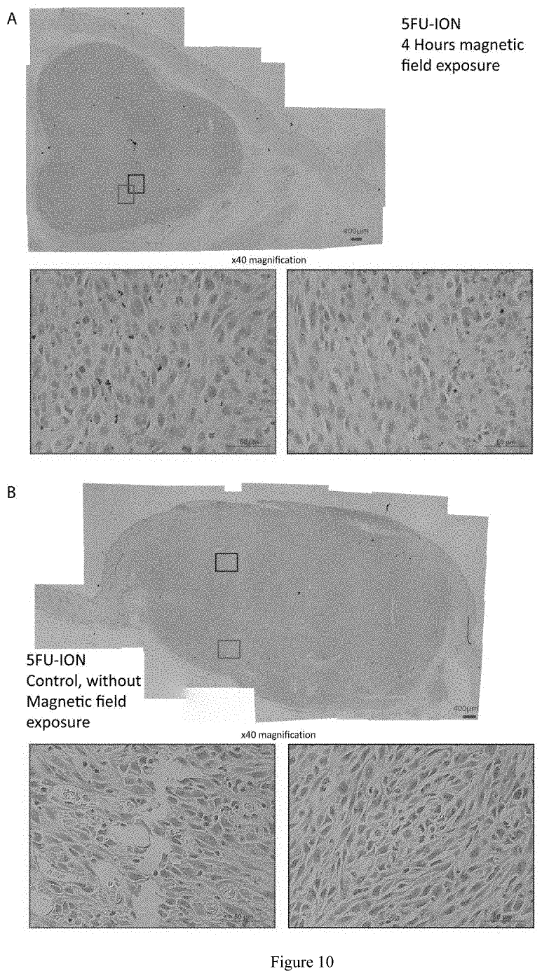

To verify that the absorbance of the 5FU, an FDA approved chemotherapeutic drug, to the IONPs does not affect their superparamagnetic characteristics we injected the 6×IONP-5FU locally underneath the tumor with or without exposure to an external magnetic field for 4 or 6 hours. In all experimental groups, exposed to a magnetic field for 4 and 6 hours, the 6×IONP-5FU were highly stained underneath the tumor suggesting the magnetic field inhibits the clearance of the IONPs. At 2 and 4 hours, the iron staining was visible also in tumor margins and in several intra-tumor regions. In the control group without magnetic field exposure, there was a lower iron staining in the margins of the tumor and almost none in regions inside the tumor (FIG. 10).

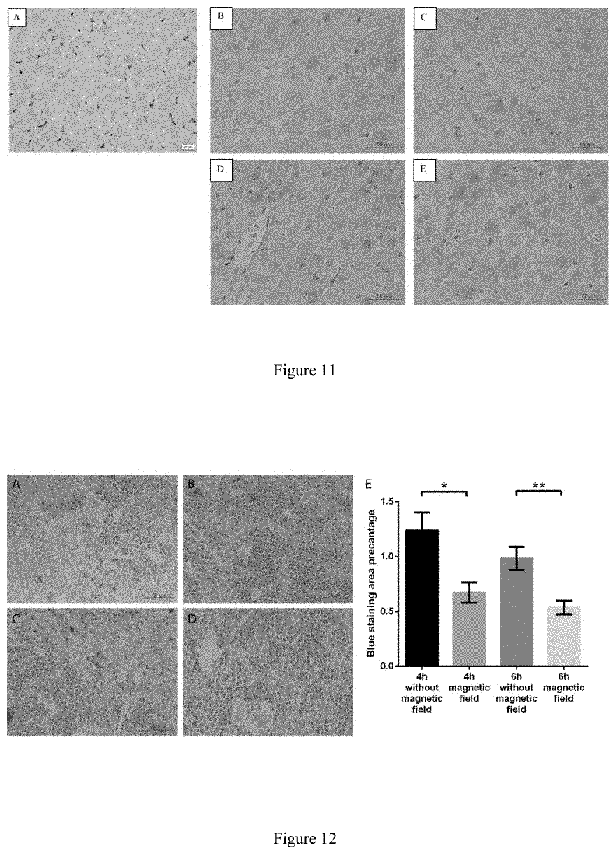

Systemic administration of 4×IONP-HSA and exposure to a magnet for 4 and 24 hours yielded higher iron staining in the spleen and liver as opposed to slightly milder staining in mice exposed to a magnetic field for 2 hours. The mice treated locally with 6×IONP-5FU with or without magnetic field exposure, showed a considerably lower iron staining in the liver (FIG. 11) suggesting local treatment reduces clearance of nanoparticles and liver toxicity.

Locally administration of 6×IONP-5FU and exposure to magnetic field yielded lower iron staining in the spleen as opposed to controls without magnetic field exposure. The average iron staining percentage in the spleens of mice exposed to a magnetic field for 4 and 6 hours was 1.83 times (P<0.05, P<0.01 respectively) higher than the spleens of mice that were not exposed to a magnetic field (FIG. 12). These results indicate less clearance of the nanoparticles caused by the magnetic field.

### Example 11

### Anti-Tumor Activity of 5FU-IONP in Tumor-Xenografted Mice

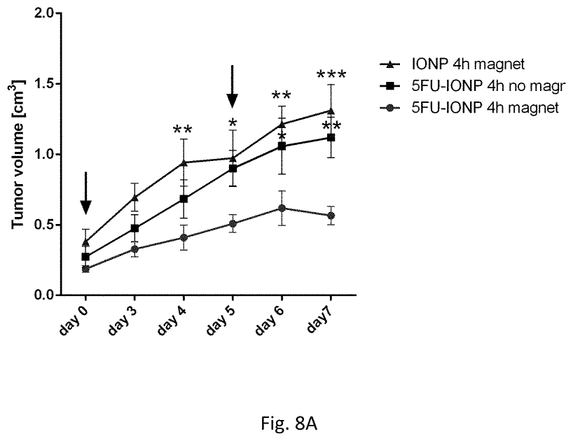

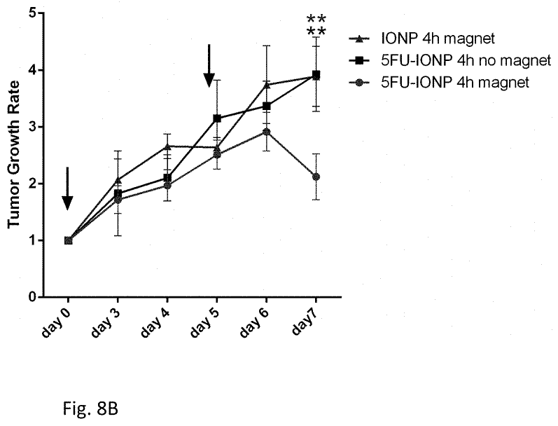

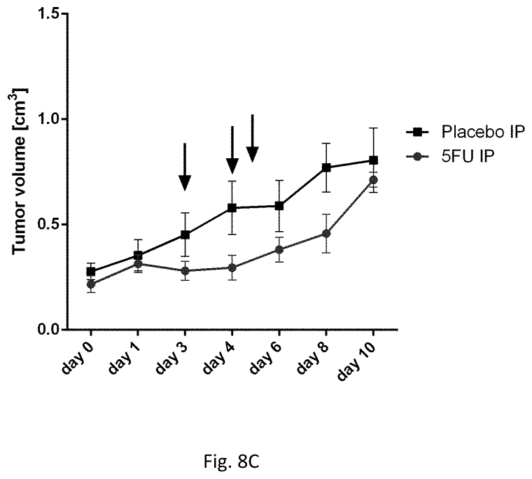

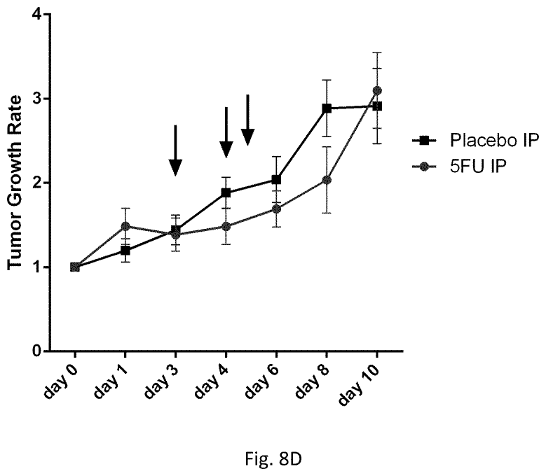

Twenty tumors bearing mice were randomly allocated to 4 groups (see Materials and methods 2.3). At days 0 and 5, 6×IONP-5FU or 6×IONP (with no 5FU) were injected underneath the tumors of 20 mice, with or without exposure to an external magnetic field for 4 hours. The tumor volumes were measured at days 3,4,5,6,7. The average tumor volume of all mice at day 0 was 0.29 cm³±0.008 cm³ with no statistically significant difference between the groups. At day 6 and 7 the average tumor volumes of experimental group treated with 6×IONP-5FU exposed to a magnetic field for 4 hours were significantly lower (0.62 cm³±0.122 cm³ and 0.57 cm³±0.144 cm³ respectively, n=5) than the average tumor volumes of the control group treated with 6×IONP-5FU without exposure to a magnetic field (1.06 cm³±0.198 cm³ P<0.05 and 1.12 cm³±0.143 cm³ P<0.01 respectively). The average tumor volumes of the experimental-1 group at days 6 and 7 were also significantly lower than the control group treated with 6×IONP alone (1.21 cm³±0.128 cm³ P<0.01 and 1.31 cm³±0.183 cm³ P<0.001 respectively, n=5) suggesting the 6×IONP alone do not possess anti-tumor effect (FIG. 8).

A comparative study of systemic treatment was performed using three consecutive intraperitoneal injections of 5FU (not bound to nanoparticles). No significant changes in tumor volumes compared to control mice treated with normal saline were shown (FIG. 8). Taking together both experiments results, we can conclude that the external magnetic field with local injection of 5FU-IONP had the best anti-tumor effect (measured by the tumor volumes).

This data suggests that exposure to an external magnetic field following local injection of chemotherapy loaded IONPs increases drug targeting to the tumor, with an anti-tumor effect characterized by inhibition of tumor growth.

### Example 12

### Anti-Tumor Activity of Bevacizumab (Bev) Superparamagnetic Iron Oxide Nanoparticles (SPIONs) in Bev Resistant Tumors

A magnetic targeting of drug-bounded superparamagnetic iron oxide nanoparticles (SPIONs) study was conducted. These SPIONs, had a diameter of 70 nm with narrow size distribution. These SPIONs were coated with human serum albumin (HSA) wherein 5FU and/or Bevacizumab (Bev) was physically absorbed on a HSA outer layer .

Ectopic CRC tumors induced on the hind limb of mice via subcutaneous (SC) injection of CT26 cells (Bev resistant).

In order to increase targeting efficiency, the approach presented in Example 2 and FIG. 6 was modified to injecting SPIONs loaded with 5FU and/or Bev (Avastin), locally into the surrounding of the tumor tissue—peritumoral tissue, rather than systemically.

By applying an external magnetic field focused on the tumor site, these locally injected SPIONs were held within the tumor surroundings, inhibiting their migration to neighboring tissues and/or systemic distribution while enhancing tumor penetration. As such, the injection of the particles directly into the peritumoral tissue appears particularly promising as the need to counterbalance the blood drag force is eliminated.

This concept of magnetic targeting was studied by local injection of SPIONs on the CT26 tumor mice model (Bev resistant, FIG. 7). Magnetic targeting (SPIONs+Mag) revealed much higher iron staining surrounding the tumor, while in the control group there was minimal staining with pseudo-magnet attachment (SPION+PMag,). Furthermore, it was demonstrated that there was a significant iron staining inside the tumor, while there was no staining in the tumors of the control groups.

The biodistribution and clearance of the SPIONs were assessed by examination of the livers and spleens of the mice. Those mice in the control group had more uptake of the iron in their spleens then the experimental group who had less, suggesting that the magnetic field maintained the SPION in the vicinity of the tumor, and inhibited their clearance (FIG. 12).

Following the verification of this method, 5FU or bev were conjugated to the SPIONs and it was demonstrated that the new complex of SPION-5FU did not change the magnetic properties of the particle complex.

This study demonstrated that while systemic injection of 5FU did not inhibit CT26 tumor growth at all, a low dose magnetic targeting of SPION-5FU (SPION-SFU+Mag) significantly inhibited tumor growth by 50% compared to mice treated with SPION-5FU with pseudo-magnet (SPION-5FU+PMag). Importantly, this study enabled the use of Bev as an effective therapy in Bev resistant tumors.

Bev is Effective only if Administered Via Magnetic Targeting

Surprisingly, even though CT26 cells were considered to be nonresponsive to Bev, magnetic targeting of SPION-Bev+Mag revealed high potency, as no statistically-significant tumor growth was evident in this group, while the tumors of the control group (SPION-Bev+PMag) continued growing.

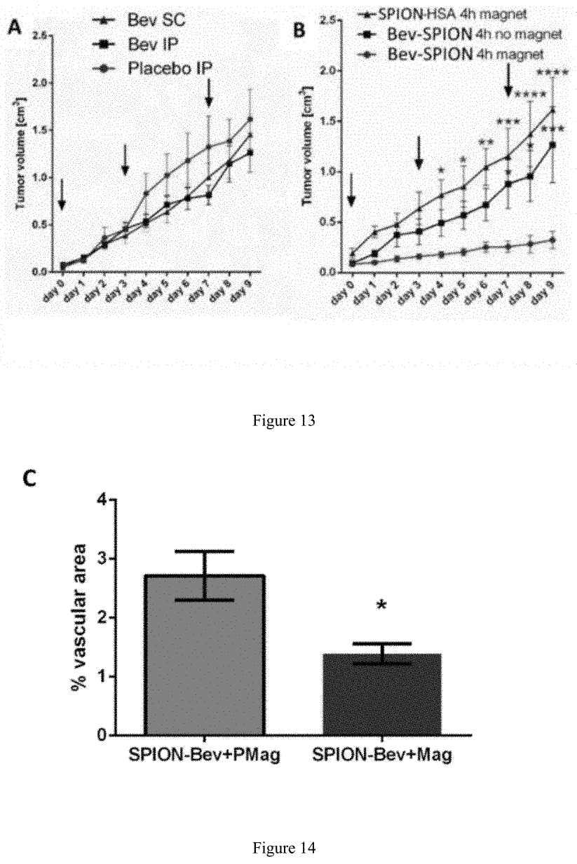

The average tumor volumes of the experimental group were significantly lower than the control group at days 7, 8 (P<0.05) and 9 (P<0.001, n=6 per group). In comparison to both standard systemic or local administration of Bev (without magnetic targeting), these routes did not yield inhibition of tumor growth (FIG. 13). However, tumors treated with SPION-BEV in the presence of a magnet had significantly stopped growing.

To determine the contribution of Bev to the inhibition of tumor growth, evaluation of vascular density was performed by immunostaining and quantification of CD31. Preliminary results show a reduction of 50% in vascular density of a tumor treated by magnetic targeting (SPION-Bev+Mag) as compared with the control group (SPION-Bev+PMag, FIG. 14).

In conclusion, local administration of drug-conjugated SPIONs and specifically Bev and SFU, with application of an external magnetic field for 2-4 hours not only resulted in increased concentration of the SPIONs in and around the tumors but actually resulted in clear tumor inhibition effect. Importantly, local administration of SPION-Bev with application of an external magnetic field surprisingly inhibited ectopic colorectal cancer (CRC) tumor growth in mice, as growth had statistically stopped. Contrary, neither standard systemic treatment with Bev nor local injection of Bev presented inhibition of tumor growth. Therefore, Bev was found to be an effective medicament for the treatment of a primary tumor within the context of the present magnetic NPs via magnetic targeting.

1. (canceled)

2. (canceled)

3. A method for inhibiting growth of a bevacizumab (Bev) non-responsive solid tumor or CRC tumor, comprising: (a) diagnosing a tumor in a subject afflicted with cancer as said Bev non-responsive solid tumor; (b) locally administering to the Bev non-responsive solid tumor or to the tumor's peritumoral tissue a superparamagnetic nanoparticle comprising Bev; and (c) applying a magnetic field on the tumor.

4. (canceled)

5. (canceled)

6. The method of claim 3, wherein the magnetic field strength is in the range 0.001 to 4 Tesla.

7. The method of claim 3, wherein the magnetic field has a magnetic field gradient of greater than 1 T/m.

8. The method of claim 3, wherein the magnetic field is a pulsating, an oscillating or a pulsating-oscillating magnetic field.

9. (canceled)

10. The method of claim 3, wherein the magnetic field is applied prior to said administering said nanoparticle.

11. The method of claim 3, wherein said magnetic field is applied in a frequency band from 10¹⁰ Hz to 10²⁰ Hz.

12. The method of claim 3, wherein said magnetic field is applied for 2 to 8 hours.

13. (canceled)

14. The method of claim 3, wherein said nanoparticle comprises a plurality of nanoparticles, wherein said magnetic field induces contact between al least 40% of said nanoparticles and said peritumoral tissue, said solid tumor, said colorectal tumor, or any combination thereof.

15. (canceled)

16. The method of claim 3, wherein said solid tumor is a rectal cancer tumor, an esophageal cancer tumor, a pancreatic cancer tumor, a prostate cancer tumor, a colon cancer tumor, or a liver cancer tumor.

17. (canceled)

18. (canceled)

19. The method of claim 3, wherein said administering is locally administering to a location which is 0.1 cm to 5 cm away from a surface of said target tissue or said peritumoral tissue.

20. (canceled)

21. (canceled)

22. A nanoparticle comprising: a core comprising a metal chelating polymer, said metal chelating polymer is coated with at least two layers of magnetic metal oxide, said at least two layers of magnetic metal oxide are further coated with a protein layer, said nanoparticle comprises bevacizumab (Bev), Fluorouracil (5-FU), or a combination thereof.

23. The nanoparticle of claim 22, wherein said polymer has functional groups capable of binding metal ions selected from amino, hydroxyl, carboxylate, —SH, ether, imine, phosphate or sulfide groups.

24. The nanoparticle of claim 22, wherein said polymer is selected from gelatin, polymethylenimine, chitosan or polylysine.

25. The nanoparticle of claim 22, wherein said magnetic metal oxide is an iron oxide or a ferrite derived from an iron oxide.

26. (canceled)

27. (canceled)

28. The nanoparticle of claim 22, wherein said polymer is gelatin, said magnetic metal oxide is iron oxide.

29. The nanoparticle of claim 22, wherein said bevacizumab (Bev), Fluorouracil (5-FU), or a combination thereof is: (a) physically or covalently bound to the outer surface of the magnetic metal oxide, (b) physically or covalently bound to the outer surface of the protein layer, (c) physically or covalently bound to the metal chelating polymer, or a combination thereof.

30. The nanoparticle of claim 22, wherein said protein layer comprises serum albumin.

31. (canceled)

32. (canceled)

33. A pharmaceutical composition comprising a nanoparticle according to claim 22 and a pharmaceutically acceptable carrier.

34. (canceled)

35. (canceled)

36. A kit or a system comprising a pharmaceutical composition comprising the nanoparticle of claims 22 and a magnetic field generating magnet, wherein said nanoparticle comprises bevacizumab, Fluorouracil (5-FU), or a combination thereof wherein said magnetic field's strength is adapted to the magnetization capacity of said nanoparticle.

37. (canceled)
